SEQUENCE LISTING (12 sequences)

SEQ ID NO: 1 - protein, 31 aa, Homo sapiens
  VARIANT (31)...(31): Valine or Alanine
SETTTGTSSNSSQSTSNSGLAPNPTNATTKX

SEQ ID NO: 2 - protein, 30 aa, Homo sapiens
SETTTGTSSNSSQSTSNSGLAPNPTNATTK

SEQ ID NO: 3 - protein, 27 aa, Mus musculus
NQTSVAPFPGNQNISASPNPTNATTRG

SEQ ID NO: 4 - protein, 26 aa, Homo sapiens
MGRAMVARLGLGLLLLALLLPTQIYS

SEQ ID NO: 5 - protein, 287 aa, Artificial
  Fusion protein
MGRAMVARLGLGLLLLALLLPTQIYSSETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 261 aa, Artificial
  Fusion protein
SETTTGTSSNSSQSTSNSGLAPNPTNATTKPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 288 aa, Artificial
  Fusion protein
MGRAMVARLGLGLLLLALLLPTQIYSSETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 288 aa, Artificial
  Fusion protein
MGRAMVARLGLGLLLLALLLPTQIYSSETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 29 aa, Macaca fascicularis
TVTTSAPLSSNSPQNTSTTPNPANTTTKA

SEQ ID NO: 11 - protein, 262 aa, Artificial
  Fusion protein
SETTTGTSSNSSQSTSNSGLAPNPTNATTKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 262 aa, Artificial
  Fusion protein
SETTTGTSSNSSQSTSNSGLAPNPTNATTKAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0046154 A1
(43) Pub. Date: Feb. 18, 2021

(54) METHODS OF USE OF SOLUBLE CD24 FOR TREATING ACQUIRED IMMUNE DEFICIENCY SYNDROME (HIV/AIDS)

(71) Applicants: ONCOIMMUNE, INC., Rockville, MD (US); UNIVERSITY OF NORTH CAROLINA AT CHAPEL HILL, Chapel Hill, NC (US); INSTITUTE OF BIOPHYSICS, CHINESE ACADEMY OF SCIENCES, Beijing (CN); KUNMING INSTITUTE OF ZOOLOGY, CHINESE ACADEMY OF SCIENCES, Yunnan (CN)

(72) Inventors: Yang Liu, Baltimore, MD (US); Pan Zhang, Baltimore, MD (US); Lishan Su, Chapel Hill, NC (US); Yong-Tang Zheng, Kunming (CN); Liguo Zhang, Beijing (CN)

(73) Assignees: ONCOIMMUNE, INC, Rockville, MD (US); UNIVERSITY OF NORTH CAROLINA AT CHAPEL HILL, Chapel Hill, NC (US); INSTITUTE OF BIOPHYSICS, CHINESE ACADEMY OF SCIENCES, Beijing (CN); KUNMING INSTITUTE OF ZOOLOGY, CHINESE ACADEMY OF SCIENCES, Yunnan (CN)

(21) Appl. No.: 16/977,628

(22) PCT Filed: Mar. 5, 2019
(86) PCT No.: PCT/US19/20712

§ 371 Date: Sep. 2, 2020

Related U.S. Application Data
(60) Provisional application No. 62/638,772, filed on Mar. 5, 2018.

(51) Int. Cl. A61K 38/17; A61P 31/18; C12N 15/86

(52) U.S. Cl. CPC A61K 38/17; C12N 15/86; A61P 31/18

(57) ABSTRACT

The present invention relates to a method of treating, mitigating, minimizing, or preventing HIV-1/AIDS by administering a CD24 protein to a subject in need thereof. Also provided herein is use of a CD24 protein in the manufacture of a medicament for treating HIV-1/AIDS. Further, provided is a pharmaceutical composition comprising a pharmaceutically acceptable amount of a CD24 protein.

### FIELD OF THE INVENTION

The present invention relates to compositions and methods for treating acquired immune deficiency syndrome (HIV/AIDS).

### BACKGROUND OF THE INVENTION

HIV-1/AIDS is one of biggest threats of global health. Although long time cART/HAART can effectively abate and maintain plasma viral load to under detectable level and partly reconstruct immune system, there are also about 20% of patients without suitable immune reconstruction [Kelley et al., 2009]. Chronic immune activation (a state of persistent and aberrant activation of immune system) is not only a characteristic of pathogenic HIV-1/SIV infection, but also a strong independent predictor of disease progression that associates with impaired immune reconstitution in HIV-1-infected individual on cART [Pallikkuth et al., 2013]. On one hand, chronic immune activation and inflammation accelerate progression of immune cells and drive them into immunosenescence through the cycle of growth and division [Deeks S G., et al., 2009]. One the other hand, ongoing chronic immune activation and inflammation form a vicious circle, boost formation of inflammatory tissue microenvironment, and finally lead to problems throughout the body which are harmful to the HIV-1 infected patient [Younas M et al., 2016; Rajasuriar et al., 2015]. Over time, persistent high level inflammation and chronic immune activation can damage organs and lead to inflammation-associated diseases which also present a high risk for serious non-AIDS conditions including cancer, cardiovascular, liver, and renal disease [Deeks et al., 2013; Rajasuriar et al., 2015]. Nowadays, cART, as well as blocking cytokine production and function, include anti-inflammatory drugs and immunosuppressants for managing chronic immune activation and inflammation to improve overall health and are important strategies for HIV-1 immune therapy [Rajasuriar et al., 2013]. Moreover, regulation of chronic immune activation and inflammation play an important role in effective therapy of other infectious diseases [Hsu et al., 2016]. Many causes have been reported to contribute to chronic immune activation and inflammation in HIV-1/SIV infection, such as the production of virus replication, co-infection or opportunistic pathogens, and products of microbial translocation [Paiardini et al., 2013]. Therapeutic strategies targeting these causes have been developed, such as valganciclovir, anti-LPS antibodies and Sevelamer carbonate with uneven effects on AIDS patients or SIV-infected animals [Hunt et al., 2011; Kristoff et al., 2014; Sandler et al., 2014]. Therefore, there remains a large unmet medical need for treating HIV-1/AIDS by controlling chronic immune activation.

### SUMMARY OF THE INVENTION

Provided herein is a method of treating, mitigating, minimizing, or preventing HIV-1/AIDS by administering a CD24 protein to a subject in need thereof. Also provided herein is use of a CD24 protein in the manufacture of a medicament for treating HIV-1/AIDS. The CD24 protein may comprise a mature human CD24 polypeptide or a variant thereof. The mature human CD24 polypeptide may comprise an amino acid sequence set forth in SEQ ID NO: 1 or 2. The CD24 protein may comprise any or all of the extracellular domain of human CD24. The CD24 protein may comprise the signal sequence, which may have the amino acid sequence set forth in SEQ ID NO: 4 to allow secretion from a cell expressing the protein. The signal peptide sequence may be one that is found on other transmembrane or secreted proteins, or one modified from the existing signal peptides known in the art. The CD24 protein may be soluble and/or may be glycosylated. The CD24 protein may be produced using a eukaryotic protein expression system, which may comprise a vector contained in a Chinese Hamster Ovary cell line or a replication-defective retroviral vector. The replication defective retroviral vector may be stably integrated into the genome of a eukaryotic cell.

The CD24 protein may comprise a protein tag, which may be fused at the N- or C-terminus of the CD24 protein. The protein may comprise a portion of a mammalian immunoglobulin (Ig) protein. The portion of the Ig protein may be a Fc region of the Ig protein, and the Ig protein may be human. The Fc region may comprise a hinge region and CH2 and CH3 domains of IgG1, IgG2, IgG3, IgG4, or IgA. The Fc region may also comprise the hinge region and CH2, CH3, and CH4 domains of IgM. The CD24 protein may comprise the amino acid sequence set forth in SEQ ID NO: 5, 6, 8, 9, 11, or 12. The amino acid sequence of the CD24 protein may also consist of the sequence set forth in SEQ ID NO: 5, 6, 8, 9, 11 or 12.

Further described herein are methods of controlling chronic inflammation and HIV viral loads by administering the CD24 to a subject in need thereof. As CD24Fc interacts with danger-associated molecular patterns (DAMPs) and Siglecs to attenuate inflammation, it was shown that it can protect Chinese rhesus macaques (ChRMs) with established simian immunodeficiency virus (SIV) infection. These results demonstrate that fortifying negative regulation of the innate immune response to DAMPs offers a new approach for treating HIV-infected patients. To substantiate these observations, the effect of CD24Fc was also tested on HIV-infected humanized mice and the data demonstrate that CD24Fc significantly reduces the production of inflammatory cytokines and immune activation of human T cells. Furthermore, CD24Fc significantly increases hematopoiesis of human stem cells in HIV-infected mice.

### BRIEF DESCRIPTION OF THE DRAWINGS

FIGS. 1A-C show the amino acid composition of the full length CD24 fusion protein, CD24Fc (also referred to herein as CD24Ig) (SEQ ID NO: 5). The underlined 26 amino acids are the signal peptide of CD24 (SEQ ID NO: 4), which are cleaved off during secretion from a cell expressing the protein and thus missing from the processed version of the protein (SEQ ID NO: 6). The bold portion of the sequence is the extracellular domain of the mature CD24 protein used in the fusion protein (SEQ ID NO: 2). The last amino acid (A or V) that is ordinarily present in the mature CD24 protein has been deleted from the construct to avoid immunogenicity. The non-underlined, non-bold letters are the sequence of IgG1 Fc, including the hinge region and CH1 and CH2 domains (SEQ ID NO: 7). FIG. 1B shows the sequence of CD24^{v}Fc (SEQ ID NO: 8), in which the mature human CD24 protein (bold) is the valine polymorphic variant of SEQ ID NO: 1. FIG. 1C shows the sequence of CD24^{A}Fc (SEQ ID NO: 9), in which the mature human CD24 protein (bold) is the alanine polymorphic variant of SEQ ID NO: 1. The various parts of the fusion protein in FIGS. 1B and 1C are marked as in FIG. 1A and the variant valine/alanine amino acid is double underlined.

FIG. 2 shows amino acid sequence variations between mature CD24 proteins from mouse (SEQ ID NO: 3) and human (SEQ ID NO: 2). The potential O-glycosylation sites are bolded, and the N-glycosylation sites are underlined.

FIGS. 3A-C. WinNonlin compartmental modeling analysis of pharmacokenitics of CD24IgG1 (CD24Fc). The opened circles represent the average of 3 mice, and the line is the predicted pharmacokinetic curve. FIG. 3A. i.v. injection of 1 mg CD24IgG1. FIG. 3B. s.c. injection of 1 mg CD24IgG1 (CD24Fc). FIG. 3C. Comparison of the total amounts of antibody in the blood as measured by areas under curve (AUC), half-life and maximal blood concentration. Note that overall, the AUC and Cmax of the s.c. injection is about 80% of i.v. injection, although the difference is not statistically significant.

FIGS. 4A-B. CD24-Siglec G (10) interaction discriminates between PAMP and DAMP. FIG. 4A. Host response to PAMP was unaffected by CD24-Siglec G(10) interaction. FIG. 4B. CD24-Siglec G (10) interaction represses host response to DAMP, possibly through the Siglec G/10-associated SHP-1.

FIGS. 5A-C. CD24 Fc binds to Siglec 10 and HMGB1 and activates Siglec G, the mouse homologue of human Siglec 10. FIG. 5A. Affinity measurement of the CD24Fc-Siglec 10 interaction. FIG. 5B. CD24Fc specifically interacts with HMGB-1 in a cation-dependent manner. CD24Fc was incubated with HMGB1 in 0.1 mM of CaCl₂ and MgCl₂, in the presence or absence of the cation chelator EDTA. CD24Fc is pulled down with protein G-beads, and the amounts of HMGB1, CD24Fc or control Fc is determined by Western blot.

FIG. 5C. CD24Fc activates mouse Siglec G by inducing Tyrosine phosphorylation (middle panel) and association with SHP-1 (upper panel). The amounts of Siglec G are shown in the lower panel. CD24^{-/-} spleen cells were stimulated with 1 μg/ml of CD24Fc, control Fc or vehicle (PBS) control for 30 minutes. Siglec G was then immunoprecipitated and probed with anti-phospho-tyrosine or anti-SHP-1.

FIGS. 6A-B. CD24Fc inhibits production of TNF-α and IFN-γ by anti-CD3 activated human T cells. The human PBML were stimulated with anti-CD3 for 4 days in the presence or absence of CD24Fc and the amounts of IFN-γ and TNF-α released in the supernatant of cell culture were measured by ELISA. Data shown are means of triplicates. Error bar, SEM.

FIGS. 7A-B. CD24 inhibits inflammatory cytokine production by human macrophages. FIG. 7A. ShRNA silencing of CD24 leads to spontaneous production of TNF-α, IL-1β, and IL-6. THP1 cells were transduced with lentiviral vectors encoding either scrambled or two independent CD24 shRNA molecules. The transduced cells were differentiated into macrophages by culturing for 4 days with PMA (15 ng/ml). After washing away PMA and non-adherent cells, the cells were cultured for another 24 hours for measurement of inflammatory cytokines, by cytokine beads array. FIG. 7B. As in FIG. 7A, except that the given concentration of CD24Fc or control IgG Fc was added to macrophages in the last 24 hours. Data shown in FIG. 7A are means and S.D. from three independent experiments, while those in FIG. 7B are representative of at least 3 independent experiments.

FIGS. 8A-E. CD24Fc protects Chinese rhesus macaque from AIDS caused by SIVmac239 infection. FIG. 8A. Diagram of the experimental schedule. FIG. 8B. Weight loss of SIVmac239-infected monkeys after vehicle (left) or CD24Fc (middle) treatment. Summary data from the study are shown in the right panel. FIGS. 8C-E. CD24Fc protects SIVmac239-infected monkey against wasting syndrome (FIG. 8C), diarrhea (FIG. 8D) and AIDS morbidity and mortality (FIG. 8E). Control group (black), CD24Fc treated group (grey). Statistical significance in FIG. 8B (right) was determined by two-way repeated measures ANOVA with Bonferroni's multiple comparisons test, and the statistical significance in FIGS. 8C-E was determined using Paired t-test.

FIGS. 9A-D. CD24Fc can delay elevation of plasma viral load and decrease proviral load. FIG. 9A. Plasma viral load in control- and CD24Fc-treated monkeys. Only monkeys that survived the 32 week study period were included in the analysis. FIG. 9B. As in (FIG. 9A), except that viral load was normalized to pre-treatment levels, which is artificially defined as 1.0. FIG. 9C. Dynamics of proviral load before and after treatment. FIG. 9D. Proviral load in tissues. Control group (black), CD24Fc treated group (grey). Statistical significance in FIGS. 9A-C was determined using two-way repeated measures ANOVA with Bonferroni's multiple comparisons test. Statistical significance was determined using Student's t-test.

FIGS. 10A-D. CD24Fc can reduce inflammation in the Gut. FIG. 10A. Transcript levels of proinflammatory factors in the rectum of SIVmac239 infected monkeys that received treatment of CD24Fc (grey) or vehicle control (black). The levels of GAPDH were used as an internal control. FIG. 10B. Granulocyte infiltration in the ileum (n=11), colon (n=10) and rectum (n=10) based on the number of MPO+ cells determined by immunofluorescence. Data shown are means and S.D. Each data point is the mean of at least 5 high-power fields counted. Control group (black), CD24Fc-treated group (grey). FIG. 10C. Representative images of H&E stained sections from control- or CD24Fc-treated monkeys. FIG. 10D. Summary data of pathological scores. Control group (black), CD24Fc treated group (grey). Statistical significance in FIGS. 10A, B and D was determined using Student's t-test.

FIGS. 11A-D. CD24Fc treatment reduces HIV-1 viral load and protects CD4+ T cell from depletion in the spleen of humanized mice with acute HIV infection. FIG. 11A. The effect of CD24Fc treatment on plasma HIV-1 loads in of R3A infected mice with or without CD24Fc administered by i.p. at 5 mg/kg on days 1, 8 and 15 after infection. FIG. 11B. Summary data indicated the percentages of CD4+ T cells in peripheral blood of R3A infected mice with or without CD24Fc. FIGS. 11C-D. Summary data indicating the absolute number of CD4+ T cells (FIG. 11C) and total human lymphocytes (FIG. 11D) in spleen of R3A infected mice with or without CD24Fc at the termination. Data shown as mean and s.e.m. *P<0.05, ** P<0.01 (analysis of two-tailed unpaired Student's t-test).

FIGS. 12A-C. CD24Fc treatment reduced HIV-1 replication in humanized mice with chronic HIV infection. FIG. 12A. The effects of CD24Fc treatment (5 mg/kg weekly for 6 weeks starting at week 7 post infection) on plasma HIV-1 loads. P values are shown. Data shown as mean and s.e.m. *P<0.05 (analysis of two-tailed unpaired Student's t-test). FIG. 12B. The representative dot plots show p24 expression by CD3+CD8− T cells from lymph nodes and spleen in 4 groups comprising mock, HIV-1, HIV-1 with CD24Fc treatment and HIV-1 with cART treatment, respectively. Numbers show the percentages of p24+ cell subsets. FIG. 12C. Summary data indicate the proportion of p24+ T cell subsets in the 4 groups from FIG. 12B. Each dot represents one mouse. P values are shown <0.05 (analysis of two-tailed unpaired Student's t-test).

FIGS. 13A-B. CD24Fc treatment significantly increased the naïve T cell proportion in humanized mice with chronic HIV infection. FIG. 13A. The representative dot plots indicate the distribution of naïve and memory CD4+ and CD8+ T cell subsets in 4 groups comprising mock, HIV-1, HIV-1 with CD24Fc treatment, and HIV-1 with cART treatment. Numbers show the percentages of cell subsets. FIG. 13B. Summary data indicated the percentages of CD4+ and CD8+ memory T cell subsets in the 4 groups. Data shown as mean and s.e.m. *P<0.05, **P<0.01 and ***P<0.001 (analysis of two-tailed unpaired Student's t-test).

FIGS. 14A-B. CD24Fc treatment significantly reduced over-activation of T cells in humanized mice with chronic HIV infection. FIG. 14A. The representative dot plots indicate the expression of CD38 and HLA-DR on both CD4+ and CD8+ T cell subsets in 4 groups of humanized mice receiving, respectively, mock, HIV-1, HIV-1 with CD24Fc treatment, and HIV-1 with cART. Numbers show the percentages of CD38- and HLA-DR-expression cell subsets. FIG. 14B. Summary data indicate the percentages of CD38+HLA-DR+ CD4 and CD8 T cells in the 4 groups. Data shown as mean and s.e.m. *P<0.05, **P<0.01 and ***P<0.001 (analysis of two-tailed unpaired Student's t-test).

FIGS. 15A-D. CD24Fc treatment blocked HIV-1-induced pro-inflammatory cytokine production in vitro and in vivo. THP-1 cells were infected with R3A stock with or without CD24Fc for 3 days. Then the cells were collected for RT-PCR of pre-IL-1β and IL6 mRNA, and supernatants were collected for ELISA of IL-1β. FIG. 15A. CD24Fc inhibited HIV R3A-induced IL-1β production by THP monocytic cells in vitro. FIG. 15B. CD24Fc inhibited the production of pre-IL-1β and IL6 mRNA. Data shown as mean and S.E.M. **P<0.01 as compared to mock and ##P<0.01 as compared to R3A (analysis of two-tailed paired Student's t-test). FIG. 15C. The diagram of CD24Fc treatment (5 mg/kg) in R3A-infected humanized mice (n=3 for each group). FIG. 15D. Summary data indicates the pro-inflammatory cytokine levels of plasma at 1-3 wpi of R3A acute infection, including IL-6, IL-8, IFN-γ and IL-17a. Data shown as mean and s.e.m. *P<0.05 as compared to R3A at according time.

FIG. 16. CD24Fc treatment rescues the proliferation of HSCs in vivo of humanized mice with chronic HIV-1 infection. Summary data of the colony-forming units that develop from CD34⁺ HSCs of mock mice (n=4), HIV-1-infected mice (n=5), and HIV-1-infected mice with CD24Fc treatment (n=4), respectively. CD24Fc was administered by i.p. at 5 mg/kg weekly for 6 weeks starting at week 7 post infection. Error bars, s.e. *P<0.05 (analysis of two-tailed unpaired Student's t-test). CFU-GM, colony-forming unit-granulocyte, macrophage. CFU-E, colony-forming unit-erythroid. CFU-GEMM, colony-forming unit-granulocyte, erythroid, macrophage, megakaryocyte.

FIG. 17 shows a plot of mean plasma CD24Fc concentration (±SD) by treatment for a PK Evaluable Population in human subjects. PK=pharmacokinetic; SD=standard deviation.

FIG. 18 shows a dose proportionality plot of CD24Fc Cₘₐₓ versus dose for a PK Evaluable Population.

FIG. 19 shows a dose proportionality plot of CD24Fc AUC_{0-42d} versus dose for a PK Evaluable Population.

FIG. 20 shows a dose proportionality plot of CD24Fc AUC_{0-inf} versus dose for a PK Evaluable Population.

### DETAILED DESCRIPTION

The inventors have discovered that, surprisingly, a soluble form of CD24 is highly effective for treating HIV-1/AIDS. The effect may be mediated through DAMPs. Pattern recognition is involved in inflammatory response triggered by both pathogen-associated and tissue damage-associated molecular patterns, respectively called PAMPs and DAMPs. The inventors have realized that recent studies have demonstrated that an exacerbated host response to DAMPs may play a part in the pathogenesis of inflammatory and autoimmune disease. DAMPs were found to promote the production of inflammatory cytokines and autoimmune diseases and in animal models, and inhibitors of DAMPs such as HMGB1 and HSP90 were consequently found to ameliorate rheumatoid arthritis (RA) (4-6). TLRs, RAGE-R, DNGR (encoded by Clec9A), and Mincle have been shown to be receptors responsible for mediating inflammation initiated by a variety of DAMPs (2, 7-14).

The inventors' recent work demonstrated that CD24-Siglec G interactions discriminate between innate immunity to DAMPs and that from PAMPs (15, 16). Siglec proteins are membrane-associated immunoglobulin (Ig) superfamily members that recognize a variety of sialic acid-containing structures. Most Siglecs have an intra-cellular immune-tyrosine inhibitory motif (ITIM) that associates with SHP-1, -2 and Cbl-b to control key regulators of inflammatory responses. The inventors have reported CD24 as the first natural ligand for a Siglec, specifically, Siglec Gin mouse and Siglec 10 in human (15). Siglec G interacts with sialylated CD24 to suppress the TLR-mediated host response to DAMPs, such as HMGB1, via a SHP-1/2 signaling mechanism (15).

Human CD24 is a small GPI-anchored molecule encoded by an open-reading frame of 240 base pairs in the CD24 gene (28). Of the 80 amino acids, the first 26 constitute the signal peptide, while the last 23 serve as a signal for cleavage to allow for the attachment of the GPI tail. As a result, the mature human CD24 molecule has only 31 amino acids. One of the 31 amino acids is polymorphic among the human population. A C to T transition at nucleotide 170 of the open-reading frame results in the substitution of alanine (A) with valine (V). Since this residue is in the immediate N-terminal to the cleavage site, and since the replacement is nonconservative, these two alleles may be expressed at different efficiencies on the cell surface. Indeed, transfection studies with cDNA demonstrated that the CD24^{v} allele is more efficiently expressed on the cell surface (28). Consistent with this, CD24^{v/v} PBL expressed higher levels of CD24, especially on T cells.

The inventors have demonstrated that CD24 negatively regulates host response to cellular DAMPs that are released as a result of tissue or organ damage, and at least two overlapping mechanisms may explain this activity. First, CD24 binds and represses host response to several DAMPs, including HSP70, HSP90, HMGB1 and nucleolin. To do this, it is presumed that CD24 may trap the inflammatory stimuli to prevent interaction with their receptors, TLR or RAGE. Second, using an acetaminophen-induced mouse model of liver necrosis and ensuring inflammation, the inventors demonstrated that through interaction with its receptor, Siglec G, CD24 provides a powerful negative regulation for host response to tissue injuries. To achieve this activity, CD24 may bind and stimulate signaling by Siglec G, whereby Siglec G-associated SHP1 triggers the negative regulation. Both mechanisms may act in concert, as mice with targeted mutation of either gene mounted much stronger inflammatory response. In fact, DC cultured from bone marrow from either CD24^{-/-} or Siglec G^{-/-} mice produced higher levels of inflammatory cytokines when stimulated with either HMGB1, HSP70, or HSP90. To the inventors' knowledge, CD24 is the only inhibitory DAMP receptor capable of shutting down inflammation triggered by DAMPs, and no drug is currently available that specifically targets host inflammatory response to tissue injuries. Furthermore, the inventors have demonstrated the ability of exogenous soluble CD24 protein to alleviate DAMP-mediated autoimmune disease using mouse models of RA, MS and GvHD.

By triggering TLRs (toll like receptors) and/or NLRs (Nod-like receptors), individually or in complex with other stimulators, DAMPs are released during necrosis, pyroptosis, secondary necrosis following apoptosis and injury. These DAMPs can drive potent innate immune responses and thus contribute, at least in part, to the chronic immune activation and systemic inflammation [Lotze et al., 2005; Chen et al., 2011]. They could have a pathogenic role in sustaining sterile inflammation, and also play an important role in disease, such as trauma, chronic inflammatory disorders, autoimmune diseases and cancer [Venereau et al., 2016; Shin et al., 2015; Kang et al., 2015]. Importantly, necrosis, pyroptosis, cell death and injury occur frequently during HIV infection and AIDS. Soluble factors from dying cells have been proposed to contribute to the systemic immune activation in response to cell damage and are also connected to microbial translocation, cell death and immune activation [Trøseid et al., 2011]. In HIV-1 infected patients, it has been demonstrated that levels of DAMPs, such as HMGB1, HSP70, and auto-reactive antibodies (Abs) increase and, although cART might reduce the levels of DAMPs, they cannot return them to normal levels [Nowak et al., 2007; Anraku et al., 2012]. Auto-reactive Abs are associated with rapid loss of naïve CD4+ T and immune cells, and high levels are also associated with rapid progression of disease [Trøseid et al., 2010; Kocsis et al., 2003; Anraku et al., 2012; Espigares et al., 2006; Agnew et al., 2003; Rawson et al., 2007; Kuwata et al., 2009]. HMGB1 can promote immune activation in complex with bacterial products via TLR signal pathways, and high levels of HMGB1 are associated with high viral load [Trøseid et al., 2013]. HMGB1 and LPS are both moderately correlated with CD38 density on CD8+ T cells in HIV-1 progressors [Trøseid et al., 2013]. Based on these data, the inventors recognized that DAMPs might play an important role in immune activation and inflammation of HIV-1 infected patients, and no drugs targeting them have been used in HIV-1/AIDS therapy.

Macrophages that express various TLRs and NLRs are important innate immune cells with phagocytosis, antigen presentation and cytokine release functions. After being triggered by PAMPs and DAMPs, or activated by stimulators, type 1 macrophages (M1) release massive amounts of proinflammatory cytokines, which can lead to immune activation, systematic inflammation and activation induced cell death. On the other hand, type 2 macrophages (M2) have high phagocytic activity, produce large amounts of anti-inflammatory cytokines and participate in tissue repair. In HIV-1 infection, virus infected CD4+ T cells undergoing apoptosis, secondary necrosis, and potentially pyroptosis, release pro-inflammatory cytokines, products of virus replication and products of microbial translocation that create a highly pro-inflammatory local environment. This polarizes macrophages toward a more inflammatory M1 phenotype, as observed in untreated AIDS patients [Sattentau and Stevenson, et al]. Therefore, there is a vicious circle among macrophage polarization, inflammation, tissue injury and, finally, disease progression. Accordingly, blocking the inflammatory activity of macrophages is a strategy for treating and preventing the progression of HIV-1/AIDS.

The inventors have demonstrated that a soluble form of CD24 protein can block the proinflammatory activity of macrophages triggered by DAMPs and protect against AIDS or death, including delayed weight loss, decreased wasting syndrome, and diarrhea. Soluble CD24 protein can also delay the increase in plasma viral load and inhibit proviral load in PBMC, marrow, and rectum without restoration of CD4+ T cell number and significant changes of T cell subsets. The inventors further discovered that soluble CD24 protein can restrain gut inflammation and decrease CD8+ T cell activation. Finally, the inventors discovered that effective soluble CD24 protein treatment correlates with effective control of sCD14 levels and moderate down-regulation of HLA-DR expression in CD8+ T cells.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated. In addition, ranges with endpoints defined by numbers recited in lists are explicitly contemplated. For example, the list 1, 2, 3, and 4 defines ranges of 1-2, 2-3, 3-4, 1-4, 1-3, and 2-4. Unless stated otherwise, the endpoints are included in such ranges.

A “peptide” or “polypeptide” is a linked sequence of amino acids and may be natural, synthetic, or a modification or combination of natural and synthetic.

“Substantially identical” may mean that a first and second amino acid sequence are at least 60%, 65%, 70%, 75%, 80%, 85%, 90%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids.

“Treatment” or “treating,” when referring to protection of an animal from a disease, means preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

A “variant” may mean a peptide or polypeptide that differs in amino acid sequence by the insertion, deletion, or conservative substitution of amino acids, but retain at least one biological activity. Representative examples of “biological activity” include the ability to bind to a toll-like receptor and to be bound by a specific antibody. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

### 2. CD24

Provided herein is a CD24 protein, which may comprise a mature CD24 polypeptide or a variant thereof. The mature CD24 polypeptide corresponds to the extracellular domain (ECD) of CD24. The mature CD24 polypeptide may be from a human or another mammal. As described above, mature human CD24 polypeptide is 31 amino acids long and has a variable alanine (A) or valine (V) residue at its C-terminal end:

(SEQ ID NO: 1)
SETTTGTSSNSSQSTSNSGLAPNPTNATTK(V/A)

The C-terminal valine or alanine may be immunogenic and may be omitted from the CD24 protein, which may reduce its immunogenicity. Therefore, the CD24 protein may comprise the amino acid sequence of human CD24 lacking the C-terminal amino acid:

(SEQ ID NO: 2)
SETTTGTSSNSSQSTSNSGLAPNPTNATTK

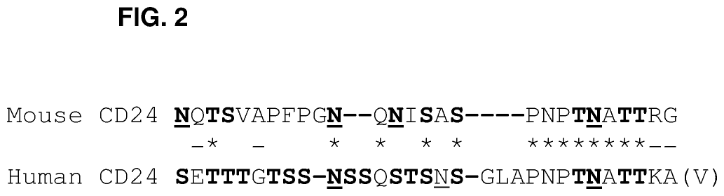

Despite considerable sequence variations in the amino acid sequence of the mature CD24 proteins from mouse and human, they are functionally equivalent, as human CD24Fc has been shown to be active in the mouse. The amino acid sequence of the human CD24 ECD shows some sequence conservation with the mouse protein (39% identity; Genbank accession number NP_033976). However, it is not that surprising that the percent identity is not higher as the CD24 ECD is only 27-31 amino acids in length, depending on the species, and binding to some of its receptor(s), such as Siglec 10/G, is mediated by its sialic acid and/or galactose sugars of the glycoprotein. The amino acid sequence identity between the extracellular domains of the human Siglec-10 (GenBank accession number AF310233) and its murine homolog Siglec-G (GenBank accession number NP_766488) receptor proteins is 63% (FIG. 2). As a result of sequence conservation between mouse and human CD24 primarily in the C-terminus and in the abundance of glycosylation sites, significant variations in the mature CD24 proteins may be tolerated in using the CD24 protein, especially if those variations do not affect the conserved residues in the C-terminus or do not affect the glycosylation sites from either mouse or human CD24. Therefore, the CD24 protein may comprise the amino acid sequence of mature murine CD24:

(SEQ ID NO: 3)
NQTSVAPFPGNQNISASPNPTNATTRG

The amino acid sequence of the human CD24 ECD shows more sequence conservation with the cynomolgus monkey protein (52% identity; UniProt accession number UniProtKB—I7GKK1) than with mouse. Again, this is not surprising given that the percent identity is not higher as the ECD is only 29-31 amino acids in length in these species, and the role of sugar residues in binding to its receptor(s). The amino acid sequence of cynomolgous Siglec-10 receptor has not been determined but the amino acid sequence identity between the human and rhesus monkey Siglec-10 (GenBank accession number XP_001116352) proteins is 89%. Therefore, the CD24 protein may also comprise the amino acid sequence of mature cynomolgous (or rhesus) monkey CD24:

(SEQ ID NO: 10)
TVTTSAPLSSNSPQNTSTTPNPANTTTKA

The CD24 protein may be soluble. The CD24 protein may further comprise an N-terminal signal peptide, to allow secretion from a cell expressing the protein. The signal peptide sequence may comprise the amino acid sequence MGRAMVARLGLGLLLLALLLPTQIYS (SEQ ID NO: 4). Alternatively, the signal sequence may be any of those that are found on other transmembrane or secreted proteins, or those modified from the existing signal peptides known in the art.

a. Fusion

The CD24 protein may be fused at its N- or C-terminal end to a protein tag, which may comprise a portion of a mammalian Ig protein, which may be human or mouse or from another species. The portion may comprise a Fc region of the Ig protein. The Fc region may comprise at least one of the hinge region, CH2, CH3, and CH4 domains of the Ig protein. The Ig protein may be human IgG1, IgG2, IgG3, IgG4, or IgA, and the Fc region may comprise the hinge region, and CH2 and CH3 domains of the Ig. The Fc region may comprise the human immunoglobulin G1 (IgG1) isotype SEQ ID NO: 7. The Ig protein may also be IgM, and the Fc region may comprise the hinge region and CH2, CH3, and CH4 domains of IgM. The protein tag may be an affinity tag that aids in the purification of the protein, and/or a solubility-enhancing tag that enhances the solubility and recovery of functional proteins. The protein tag may also increase the valency of the CD24 protein. The protein tag may also comprise GST, His, FLAG, Myc, MBP, NusA, thioredoxin (TRX), small ubiquitin-like modifier (SUMO), ubiquitin (Ub), albumin, or a Camelid Ig. Methods for making fusion proteins and purifying fusion proteins are well known in the art.

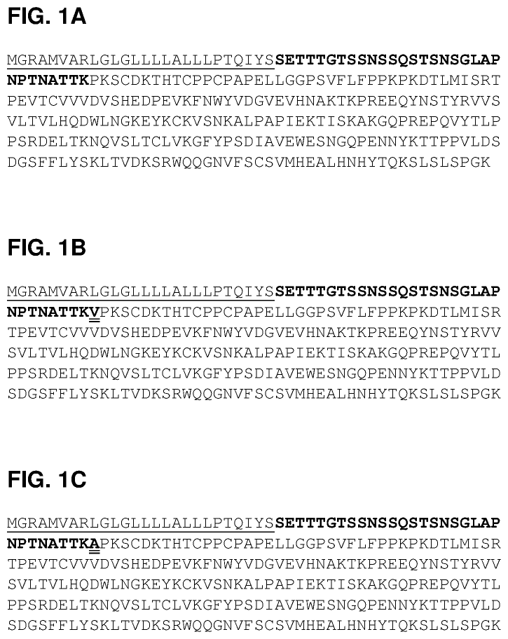

Based on preclinical research, for the construction of the fusion protein CD24Fc identified in the examples, the truncated form of native CD24 molecule of 30 amino acids, which lacks the final polymorphic amino acid before the GPI signal cleavage site (that is, a mature CD24 protein having SEQ ID NO: 2), has been used. The mature human CD24 sequence is fused to a human IgG1 Fc domain (SEQ ID NO: 7). The full length CD24Fc fusion protein is provided in SEQ ID NO: 5 (FIG. 1A), and the processed version of CD24Fc fusion protein that is secreted from the cell (i.e. lacking the signal sequence which is cleaved off) is provided in SEQ ID NO: 6. Processed polymorphic variants of mature CD24 (that is, mature CD24 protein having SEQ ID NO: 1) fused to IgG1 Fc may comprise the amino acid sequence set forth in SEQ ID NO: 11 or 12.

b. Production

The CD24 protein may be heavily glycosylated, and may be involved in functions of CD24 such as costimulation of immune cells and interaction with a damage-associated molecular pattern molecule (DAMP). The CD24 protein may be prepared using a eukaryotic expression system. The expression system may entail expression from a vector in mammalian cells, such as Chinese Hamster Ovary (CHO) cells. The system may also be a viral vector, such as a replication-defective retroviral vector that may be used to infect eukaryotic cells. The CD24 protein may also be produced from a stable cell line that expresses the CD24 protein from a vector or a portion of a vector that has been integrated into the cellular genome. The stable cell line may express the CD24 protein from an integrated replication-defective retroviral vector. The expression system may be GPEx™.

c. Pharmaceutical Composition

The CD24 protein may be contained in a pharmaceutical composition, which may comprise a pharmaceutically acceptable amount of the CD24 protein. The pharmaceutical composition may comprise a pharmaceutically acceptable carrier. The pharmaceutical composition may comprise a solvent, which may keep the CD24 protein stable over an extended period. The solvent may be PBS, which may keep the CD24 protein stable for at least 66 months at −20° C. (−15˜-25° C.). The solvent may be capable of accommodating the CD24 protein in combination with another drug.

The pharmaceutical composition may be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The pharmaceutical composition may also be formulated as a depot preparation, which may be administered by implantation or by intramuscular injection. The composition may be formulated with suitable polymeric or hydrophobic materials (as an emulsion in an acceptable oil, for example), ion exchange resins, or as sparingly soluble derivatives (as a sparingly soluble salt, for example). A formulation for subcutaneous injection may be particularly relevant for an indication like lupus and its associated manifestations and complications.

d. Dosage

The dose of the CD24 protein may ultimately be determined through a clinical trial to determine a dose with acceptable toxicity and clinical efficacy. The initial clinical dose may be estimated through pharmacokinetics and toxicity studies in rodents and non-human primates. The dose of the CD24 protein may be 0.01 mg/kg to 1000 mg/kg, and may be 1 to 500 mg/kg, depending on the desired effect on irAEs or GvHD and the route of administration. The CD24 protein may be administered by intravenous infusion or subcutaneous, intramural (that is, within the wall of a cavity or organ), or intraperitoneal injection, and the dose may be 10-1000 mg, 10-500 mg, 10-240 mg, 10-120 mg, or 10, 30, 60, 120, or 240 mg, where the subject is a human.

### 3. Methods of Treatment

a. HIV/AIDS

Provided herein is a method of mitigating or treating acquired immune deficiency syndrome (HIV/AIDS) by administering the CD24 protein to a subject in need thereof. The CD24 protein may be administered to a subject with or at risk of developing HIV/AIDS. The CD24 protein may be used prophylactically to prevent HIV/AIDS or before the clinical signs of HIV/AIDS emerge. The CD24 protein may also be administered therapeutically to treat HIV/AIDS after the clinical symptoms are diagnosed.

In another embodiment, the CD24 protein may be used to reduce or block inflammation associated with HIV/AIDS, which may comprise one or more of restraining the proinflammatory activity of macrophages triggered by DAMPs, reducing gut inflammation, decreasing CD8+ T cell activation, controlling sCD14 levels, and down-regulating HLA-DR expression in CD8+ T cells.

In another embodiment, the CD24 protein may be used to reduce or minimize the effects of HIV/AIDS, which may be one or more of weight loss, wasting syndrome, and diarrhea.

In yet another embodiment, the CD24 protein may be used to delay the increase in plasma viral load and inhibit proviral load in one or more of PBMC, marrow and rectum without restoration of CD4+ T cell number and significant changes of T cell subsets. Also provided is the use of the CD24 protein in the manufacture of a medicament for a use or treatment described herein.

b. Administration

The route of administration of the pharmaceutical composition may be parenteral. Parenteral administration includes, but is not limited to, intravenous, intraarterial, intraperitoneal, subcutaneous, intramuscular, intrathecal, intraarticular, and direct injection. The pharmaceutical composition may be administered to a human patient, cat, dog, large animal, or an avian. The composition may be administered 1, 2, 3, 4, 5, 6, 7, 8, 9, 10, 11, or 12 times per day.

c. Combination Treatment

Chronic immune activation and inflammation that are associated with HIV/AIDS progression are two of the biggest challenges for HIV-1 therapy [Appay et al., 2008]. Although successful cART can suppress plasma viral load to undetectable levels, chronic immune activation and inflammation are still not extinguished and closely associate with non-AIDS defining disease and death [Rajasuriar et al., 2015]. Currently, various kind of immunosuppressants (Predbisone, mycophenolate, Cyclosorine, Sirolimus/rapamycin), anti-inflammatory drugs (aspirin, Celecoxib, Chloroquine, Hydroxychloroquine, Pentoxifyline, Salsalate, Adalimumab, Infliximab/etanercept) [Rajasuriar et al., 2013], and statins (Atorvastatin, fluvastatin, lovastatin, pitavastatin, pravastatin, rosuvastatin, simvastatin) have been tested for anti-chronic immune activation and inflammatory effects in the clinic or animals [Eckard et al., 2015], but their effects are associated with different side effects. In particular, non-specific drugs like immunosuppressants have variable effects on viral loading, chronic immune activation and inflammation with a high risk for opportunistic infection; non-steroidal anti-inflammatory drugs have effects on anti-chronic immune activation and high risk of cardiovascular disease; and, statins, which have the benefits of controlling inflammation, immune activation, and immune senescence, also present a high risk of heart failure, myalgia, rhabdomyolysis, mental and neurological symptoms, and cancer [Ravnskov et al., 2006; Rajasuriar et al., 2013]. However, immune therapies with highly specific administration, such as anti-TNF-α antibodies, are more effective and have fewer side effects [Tabb et al., 2013]. Therefore, enhancing the specificity of the treatment may improve the efficacy of treatment with higher tolerance and lower side effects. Accordingly, the CD24 proteins described herein may be administered in combination with any of these other therapies in a method of treatment described herein.

Such combination therapies include antiretroviral therapy (ART), including highly active antiretroviral therapy (HAART) and/or combination antiretroviral therapy (cART). Examples of ART include entry inhibitors, nucleoside/nucleotide reverse transcriptase inhibitors (NRTIs), non-nucleoside reverse transcriptase inhibitors (NNRTIs), integrase inhibitors (also known as integrase nuclear strand transfer inhibitors or INSTIs), and protease inhibitors. Entry inhibitors (or fusion inhibitors) such as Maraviroc and enfuvirtide, interfere with binding, fusion and entry of HIV-1 to the host cell by blocking one of several targets, such as CCRS and CXCR4 or gp41 of HIV. NRTIs are nucleoside and nucleotide analogues, such as zidovudine, abacavir, lamivudine, emtricitabine, and tenofovir, which inhibit reverse transcription and thus integration into the host cell genome. NNRTIs also inhibit reverse transcriptase, but do so by binding to an allosteric site of the enzyme. NNRTIs include nevirapine, efavirenz, etravirine and rilpivirine. The viral enzyme integrase is responsible for integration of viral DNA into the DNA of the infected cell. Thus, integrase inhibitors, such as raltegravir, elvitegravir and dolutegravir, prevent this step in the virus replication. Protease inhibitors block the viral protease enzyme necessary to produce mature virions upon budding from the host membrane by preventing the cleavage of gag and gag/pol precursor proteins, and include lopinavir, indinavir, nelfinavir, amprenavir, ritonavir, darunavir and atazanavir. Examples of fixed dose combinations of ART that can be used in combination with the CD24 proteins include Combivir (lamivudine+zidovudine, GlaxoSmithKline), Kaletra (lopinavir+ritonavir, Abbott Laboratories), Trizivir (abacavir+lamivudine+zidovudine, GlaxoSmithKline), Epzicom/Kivexa (abacavir+lamivudine, GlaxoSmithKlinezzO, Truvada (tenofovir disoproxil fumarate+emtricitabine, Gilead Sciences), Atripla (emtricitabine+tenofovir disoproxil fumarate+efavirenz, Gilead Sciences and Bristol-Myers Squibb), Complera/Eviplera (emtricitabine+rilpivirine+tenofovir disoproxil fumarate, Gilead Sciences and Janssen Therapeutics), Stribild (elvitegravir+cobicistat+emtricitabine+tenofovir disoproxil fumarate, Gilead Sciences), Triumeq (abacavir+dolutegravir+lamivudine, ViiV Healthcare), Evotaz (atazanavir+cobicistat, Bristol-Myers Squibb), Prezcobix (darunavir+cobicistat, Janssen Therapeutics), Dutrebis (lamivudine+raltegravir, Merck & Co.), Genvoya (elvitegravir+cobicistat+emtricitabine+tenofovir alafenamide fumarate, Gilead Sciences), and Descovy (emtricitabine+tenofovir alafenamide fumarate, Gilead Sciences). Other combination therapies include valganciclovir, anti-LPS antibodies and Sevelamer carbonate.

The CD24 protein may be administered simultaneously or metronomically with other treatments. The term “simultaneous” or “simultaneously” as used herein, means that the CD24 protein and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The CD24 protein may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, and 1 mins. The CD24 protein may be administered at any point prior to a second treatment of the CD24 protein including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

The CD24 protein may be administered at any point after another treatment including about 1 min, 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, and 120 hr. The CD24 protein may be administered at any point prior after a previous CD24 treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

### Example 1

### CD24 Pharmacokinetics in Mice

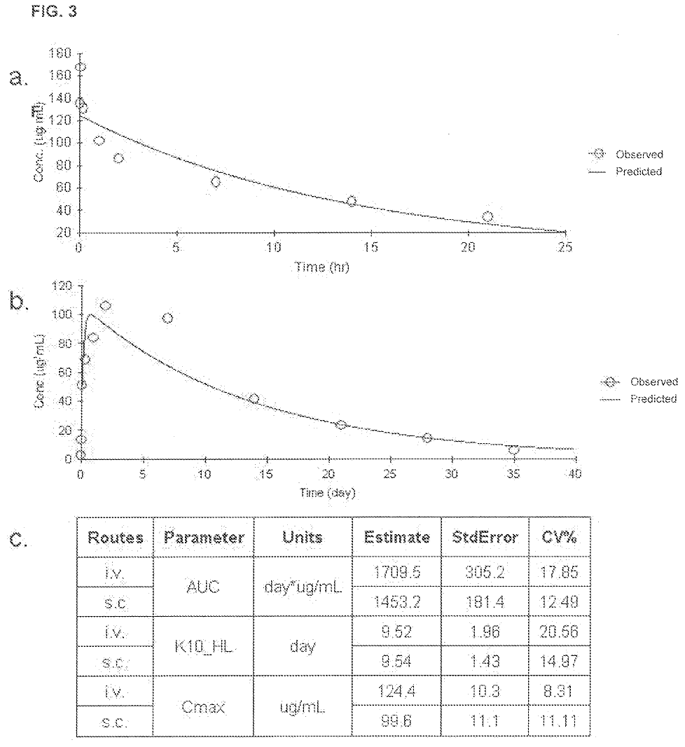

1 mg of CD24Fc (CD24Fc) was injected into naïve C57BL/6 mice and collected blood samples at different timepoints (5 min, 1 hr, 4 hrs, 24 hrs, 48 hrs, 7 days, 14 days and 21 days) with 3 mice in each timepoint. The sera were diluted 1:100 and the levels of CD24Fc was detected using a sandwich ELISA using purified anti-human CD24 (3.3 μg/ml) as the capturing antibody and peroxidase conjugated goat anti-human IgG Fc (5 μg/ml) as the detecting antibodies. As shown in FIG. 3a. The decay curve of CD24Fc revealed a typical biphase decay of the protein. The first biodistribution phase had a half-life of 12.4 hours. The second phase follows a model of first-order elimination from the central compartment. The half-life for the second phase was 9.54 days, which is similar to that of antibodies in vivo. These data suggest that the fusion protein is very stable in the blood stream. In another study in which the fusion protein was injected subcutaneously, an almost identical half-life of 9.52 days was observed (FIG. 3b). More importantly, while it took approximately 48 hours for the CD24Fc to reach peak levels in the blood, the total amount of the fusion protein in the blood, as measured by AUC, was substantially the same by either route of injection. Thus, from a therapeutic point of view, using a different route of injection should not affect the therapeutic effect of the drug. This observation greatly simplified the experimental design for primate toxicity and clinical trials.

### Example 2

### CD24-Siglec 10 Interaction in Host Response to Tissue Injuries

Nearly two decades ago, Matzinger proposed what was popularly called danger theory. In essence, she argued that the immune system is turned on when it senses the dangers in the host. Although the nature of danger was not well defined at the time, it has been determined that necrosis is associated with the release of intracellular components such as HMGB1 and Heat-shock proteins, which were called DAMP, for danger-associated molecular patterns. DAMP were found to promote production of inflammatory cytokines and autoimmune diseases. In animal models, inhibitors of HMGB1 and HSP90 were found to ameliorate RA. The involvement of DAMP raised the prospect that negative regulation for host response to DAMP can be explored for RA therapy.

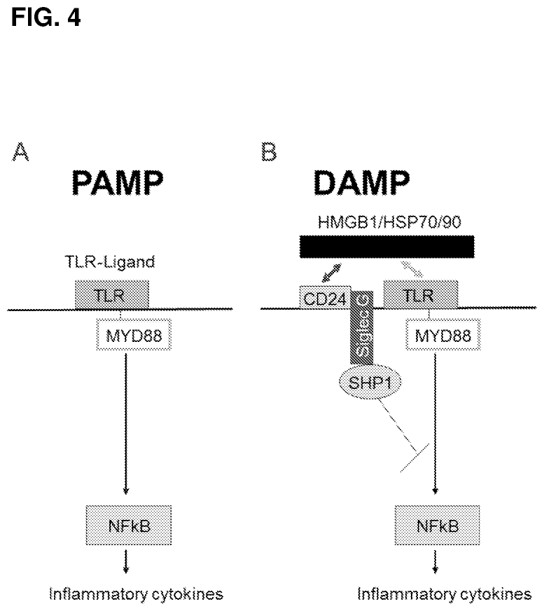

Using acetaminophen-induced liver necrosis and ensuring inflammation, it was observed that through interaction Siglec G, CD24 provides a powerful negative regulation for host response to tissue injuries. CD24 is a GPI anchored molecules that is broadly expressed in hematopoietic cells and other tissue stem cells. Genetic analysis of a variety of autoimmune disease in human, including multiple sclerosis, systemic lupus erythromatosus, RA, and giant cell arthritis, showed significant association between CD24 polymorphism and risk of autoimmune diseases. Siglec G is a member of I-lectin family, defined by their ability to recognize sialic acid containing structure. Siglec G recognized sialic acid containing structure on CD24 and negatively regulates production of inflammatory cytokines by dendritic cells. In terms of its ability to interact with CD24, human Siglec 10 and mouse Siglec G are functionally equivalent. However, it is unclear if there is a one-to-one correlation between mouse and human homologues. Although the mechanism remains to be fully elucidated, it is plausible that SiglecG-associated SHP1 may be involved in the negative regulation. These data lead to a new model in which CD24-Siglec G/10 interaction may play a critical in discrimination pathogen-associated molecular pattern (PAMP) from DAMP (FIG. 4).

At least two overlapping mechanisms may explain the function of CD24. First, by binding to a variety of DAMP, CD24 may trap the inflammatory stimuli to prevent their interaction with TLR or RAGE. This notion is supported by observations that CD24 is associated with several DAMP molecules, including HSP70, 90, HMGB1 and nucleolin. Second, perhaps after associated with DAMP, CD24 may stimulate signaling by Siglec G. Both mechanisms may act in concert as mice with targeted mutation of either gene mounted much stronger inflammatory response. In fact, DC cultured from bone marrow from either CD24−/− or Siglec G−/− mice produced much higher inflammatory cytokines when stimulated with either HMGB1, HSP70, or HSP90. In contrast, no effect were found in their response to PAMP, such as LPS and PolyI:C. These data not only provided a mechanism for the innate immune system to distinguish pathogen from tissue injury, but also suggest that CD24 and Siglec G as potential therapeutic targets for diseases associated with tissue injuries.

### Example 3

### CD24Fc Interacts with HMGB1, Siglec 10 and Induces Association Between Siglec G and SHP-1

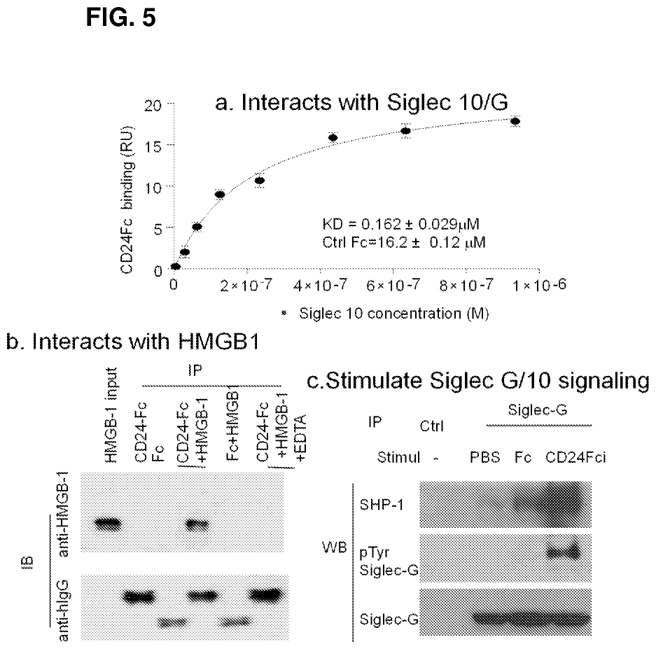

To measure the interaction between CD24Fc and Siglec 10, CD24Fc was immobilized onto a CHIP and used Biacore to measure the binding of different concentrations of Siglec-10Fc. As shown in FIG. 5a, CD24Fc binds with Siglec 10 with a Kd of 1.6×10⁻⁷M. This is 100-fold higher affinity than the control Fc. The interaction between CD24Fc and HMGB1 was confirmed by pull down experiments using CD24Fc-bound protein G beads followed by Western blot with either anti-IgG or anti-HMGB1. These data demonstrate that CD24Fc, but not Fc, binds to HMGB1 and that this binding is cation-dependent (FIG. 5b). To determine whether CD24Fc is an agonist of Siglec G, the mouse counterpart of human Siglec 10, CD24−/− spleen cells were stimulated with CD24Fc, control Fc or vehicle (PBS) control for 30 minutes. Siglec G was then immunoprecipitated and probed with anti-phospho-tyrosine or anti-SHP-1. As shown in FIG. 5c, CD24Fc induced substantial phosphorylation of Siglec G and association of SHP-1, a well-known inhibitor for both adaptive and innate immunity.

In Vitro Efficacy Studies of CD24Fc.

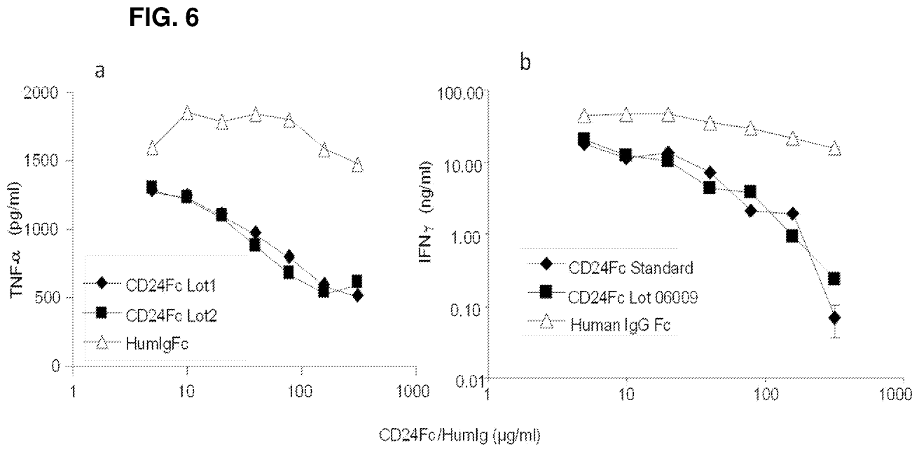

To study the impact of CD24Fc on the production of inflammatory cytokines by human T cells, the mature T cells in human PBML were activated by anti-CD3 antibody (OKT3), a commonly used agonist of the T cell receptor in the presence of different concentrations of CD24Fc or human IgG1 Fc. Four days later, the supernatants were collected and the production of IFN-γ and TNF-α were measured by Enzyme-linked immunosorbent assay (ELISA) to confirm activation. The results in FIG. 6 demonstrated that CD24Fc from two different manufacturing lots significantly reduced IFN-γ and TNF-α production from the activated human PBML compared with control IgG Fc control. In addition, when CD24Fc was added, cytokine production was inhibited in a dose-dependent manner. Therefore, CD24Fc can inhibit anti-CD3 induced human PBML activation in vitro. This study not only indicated the mechanism of action of CD24Fc might be through the inhibition of T cell activation, but also established a reliable bioassay for drug potency and stability testing.

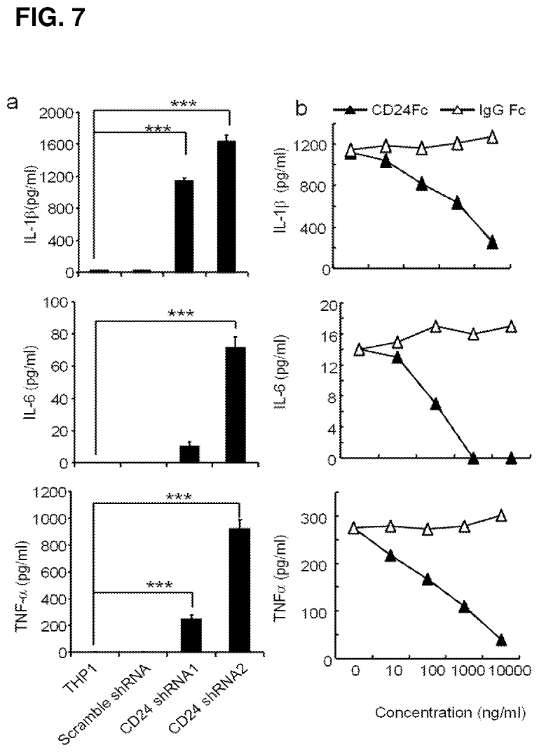

To determine whether CD24Fc regulates production of inflammatory cytokines in a human cell line, CD24 in the human acute monocytic leukemia THP1 cell line was first silenced using RNAi, and then differentiation into macrophages was induced by treating them with PMA. As shown in FIG. 7a, CD24 silencing substantially increased the production of TNFα, IL-1β and IL-6. These data demonstrate an essential role for endogenous human CD24 in limiting the production of inflammatory cytokines. Importantly, CD24Fc restored inhibition of TNFα in the CD24-silenced cell line (FIG. 7b), as well as IL-1 β and IL-6. These data not only demonstrate the relevance of CD24 in inflammatory response of human cells, but also provides a simple assay to assess biological activity of CD24Fc.

Taken together, these data demonstrate that CD24Fc is capable of inhibiting cytokine production triggered by adaptive and innate stimuli. However, since the drug is much more effective in reducing cytokine production by innate effectors, the primary mechanism for its prophylactic function was considered to be prevention of inflammation triggered by tissue injuries at the early phase of transplantation.

### Example 4

### CD24 and the Prevention of SIV

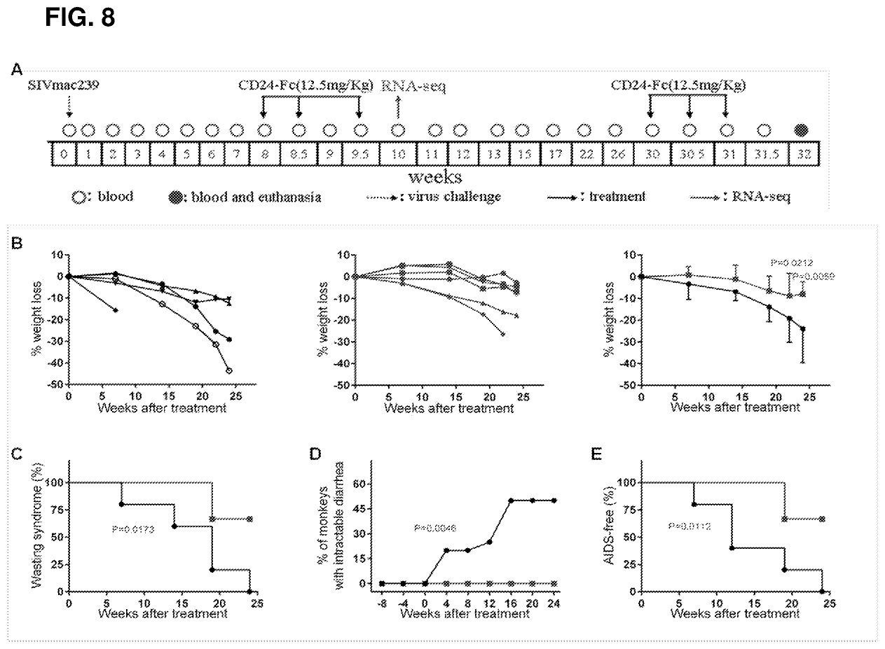

CD24Fc Protects SIV-Infected Rhesus Macaques. Two groups of SIV-infected Chinese rhesus macaques (ChRMs) were treated with either vehicle control or CD24Fc (12.5 mg/kg) on weeks 8, 8.5, 9.5, 30, 30.5 and 31 days after infection and immune activation was monitored throughout the course of the study (FIG. 8A). The body weight were measured at day 0 and then 56, 107, 155, 189, 209 and 223 days after infection (DAI). The weight loss relative to 56 DAI is shown in FIG. 8B. A very significant impact of CD24Fc on the SIV-infected monkey weight was observed. In the control group, the rates of weight loss over 10% were 25% (1/4), 75% (3/4), 75% (3/4) and 100% (4/4) on 155, 189, 209 and 231 DAI. At 107 DAI, one monkey in the control group had weight loss over 15% (15.66%) and died at 119 DAI and two control subjects had weight loss over 25% (29.11% and 43.68%). In the CD24Fc treated group the frequency of monkeys with weight loss over 10% during 155, 189, 209 and 231 DAI were 0 (0/6), 33.33% (2/6), 20% (1/5) and 20% (1/5), respectively, on those dates. One subject with the lowest CD4+ T counts at the start of treatment died at 207 DAI. Using loss of at least 10% of body weight as the basis for AIDS wasting syndrome, CD24Fc treatment significantly decreased AIDS wasting syndrome (P=0.0173) (FIG. 8C).

Diarrhea is another common symptom in HIV-1/AIDS associated with gastrointestinal dysfunctional and opportunistic infection. The health status of the monkeys was checked every day and recorded. If persistent diarrhea was observed for two days, the diagnosis was confirmed and the monkeys received treatment with penicillin. If the symptoms did not remit after 3 days of treatment, selectrin was used and the dose of penicillin was increased. If the symptoms persisted after one week's treatment, this was diagnosed as an intractable diarrhea. As shown in FIG. 8D, in the control group three monkeys had intractable diarrhea and one of them died from intractable diarrhea after 2 weeks, and the others had weight loss over 25%. Monkeys assigned to the CD24Fc group had diarrhea prior to treatment but recovered quickly. Two monkeys developed diarrhea at 4 weeks after CD24Fc treatment, but soon recovered. Therefore, CD24Fc protected all monkeys from developing intractable diarrhea. Using a log-rank test, a statistically significant difference in rate of diarrhea was found between the CD24Fc and control groups (P=0.0046). As wasting syndrome and intractable diarrhea are the most common syndromes in AIDS, the subjects with either or both symptoms were considered to have succumbed to AIDS. Using Kaplan Meier analysis, a statistically significant protection against AIDS by CD24Fc was found (P=0.0112) (FIG. 8E).

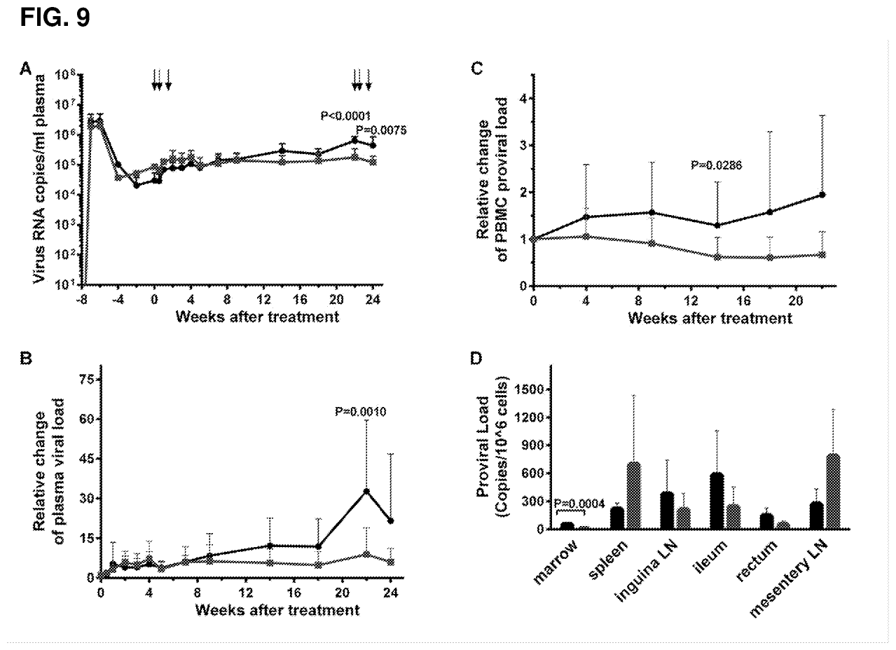

CD24Fc Delayed Elevation of Plasma Viral Load and Decreased Proviral Load in PBMC, Marrow and the Gut. To evaluate the effects of CD24Fc on virus replication, viral load in plasma and proviral load in tissues were detected. SIV infection is characterized by a rapid rise of plasma viral load, quickly followed by viral loads falling to the lowest levels. Since the goal was to study the impact of attenuating inflammation after viral replication was largely under control, the treatment at 8 weeks after infection, when the viral titer is at lowest level, was initiated. As expected, the plasma viral load increased gradually in the control group. Surprisingly, very little increase in viral load was observed in the CD24Fc treated group, resulting in a significant reduction in viral load at 26 and 30 weeks when compared with pre-treatment levels (FIG. 9A). When compared with 8 weeks after infection (normalized as 1.0), maximal increase in viral load in the CD24Fc treated group (8.79±4.54) was observed at 30 weeks after infection, which is significantly lower than that observed in the control group (32.68±13.45) (P=0.001; FIG. 9B). Furthermore, CD24Fc also appeared to have reduced proviral load in the PBMC at all time-points tested (FIG. 9C), although this reduction was not statistically significant. When tissue proviral loads were compared, the CD24Fc treatment group had significantly lower levels in bone marrow (P=0.0004), which is known to be a major virus reservoir (FIG. 9D).

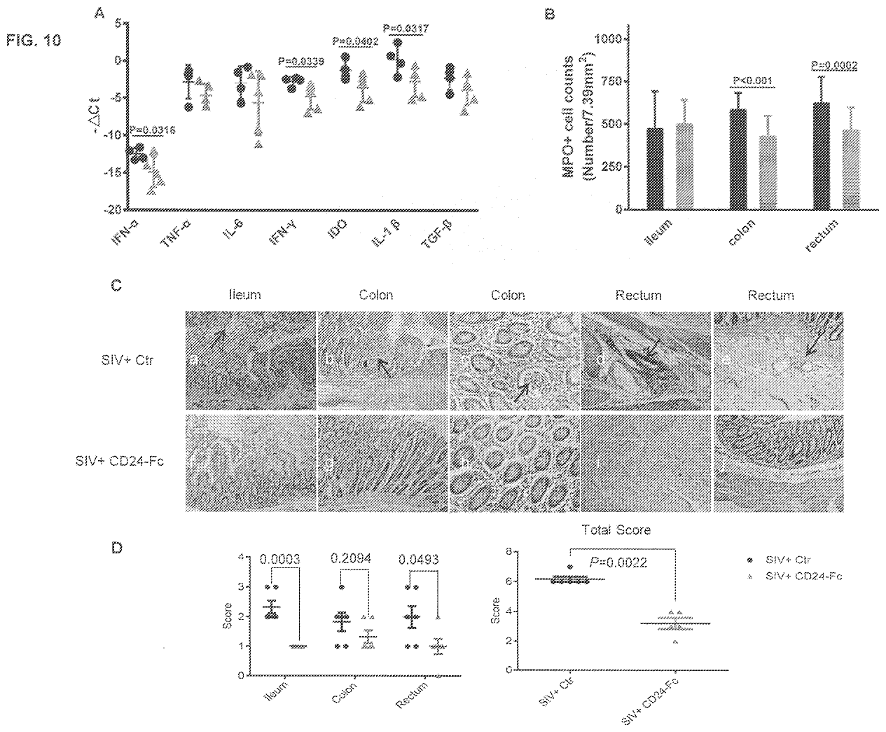

CD24Fc Can Reduce Inflammation in the Intestinal Tract. The effect of CD24Fc on inflammation was assessed using the expression of inflammation factors in SIV-infected monkeys. Unexpectedly, CD24Fc had no effect on IFN-α, TNF-α, IL-6, IFN-γ, IDO, and IL-1β expression in PBMCs in a longitudinal analysis. Therefore, systemic reduction of inflammatory cytokines may not explain the therapeutic effect of CD24Fc. To address the impact of CD24Fc on the inflammatory response of internal organs, another round of CD24Fc treatment was initiated at 30 weeks (12.5 mg/kg×3 on weeks 30, 30.5 and 31 weeks) after SIV infection, and monkeys were euthanatized at 32 weeks after infection to analyze the transcripts of inflammatory cytokines and pathology in the intestinal tract. No effect of CD24Fc on IFN-α, TNF-α, IL-6, IFN-γ, IDO, or IL-1β expression was observed in the spleen, marrow, mesentery LN, inguinal LN or ileum LCs (data not shown). However, CD24Fc treatment had attenuated expression of TNF-α, IFN-γ, IDO, and IL-1β in the rectum (FIG. 10A). These results implied that CD24Fc treatment can selectively depress gut inflammation. To corroborate these data, granulocyte infiltration was analyzed through immunofluorescence staining of MPO expression in the ileum, colon and rectum. Although MPO positive cells were detected in all sections of individuals from the control or CD24Fc treatment groups, the CD24Fc treated group had significant lower numbers of MPO+ cells in the rectum and colon than control group (FIG. 10B).

Inflammatory cell infiltration, epithelial changes and mucosal architecture were defined as the three main categories for gut pathology [83] [Geboes et al., 2000]. Generally, leukocyte density and expansion of leukocyte infiltration are two criteria for inflammatory cell infiltration. Epithelial changes include crypt epithelial cell hyperplasia, the loss of goblet cells, as well as cryptitis and crypt abscesses. Mucosal architecture was graded based on the presence of ulcerations, irregular crypts or granulation tissue. Histopathological analysis of sections from the intestinal tract was performed, and the sections were scored in a double-blinded manner. Representative images of H&E staining are shown in FIG. 10C and the summary scores are presented in FIG. 10D. Histological examination of the small intestine, colon and rectum showed the breakdown of intact epithelial barrier, the detachment of glandular epithelial cells to the lumen in ileum section (FIG. 10Ca), the intraepithelial and interepithelial abscess formation with marked neutrophil, macrophage and eosinophil infiltration in colon sections (FIG. 10Cb), muscularis perivascular lymphocytic infiltration (FIG. 10Cd) and interstitial edema (FIG. 10Ce) in rectum sections. The pathologic changes demonstrated severe inflammation in the control SIV-infected group. In contrast, the CD24Fc treated group showed only mild epithelial detachment in ileum (FIG. 10Cf). There was no cryptitis or crypt abscess in colon sections from the CD24Fc treated group, although the cryptic hyperplasia was present (FIG. 10Cg). There was minimal lymphocyte infiltration in the rectal muscularis layer and minimal interstitial edema (FIG. 10Ci). When the pathology scores are combined, it is clear that CD24Fc dramatically reduced the gut inflammation in SIV-infected monkeys.

In conclusion, these data suggest that CD24Fc can reduce large intestinal inflammation, immune activation and regulate SIV specific CD4⁺ T cell responses and T cell proliferation, and CD24Fc administration may be beneficial to SIV infected animals. This study also highlights the importance of DAMPs in the pathogenesis of HIV-1 infection and demonstrates that blocking innate immune responses triggered by DAMPs is an immune therapeutic strategy for the control/treatment of HIV-1/AIDS, and that CD24Fc is a potential therapeutic agent for AIDS therapy.

### Example 5

### CD24 Treatment of HIV Infection in Humanized Mice

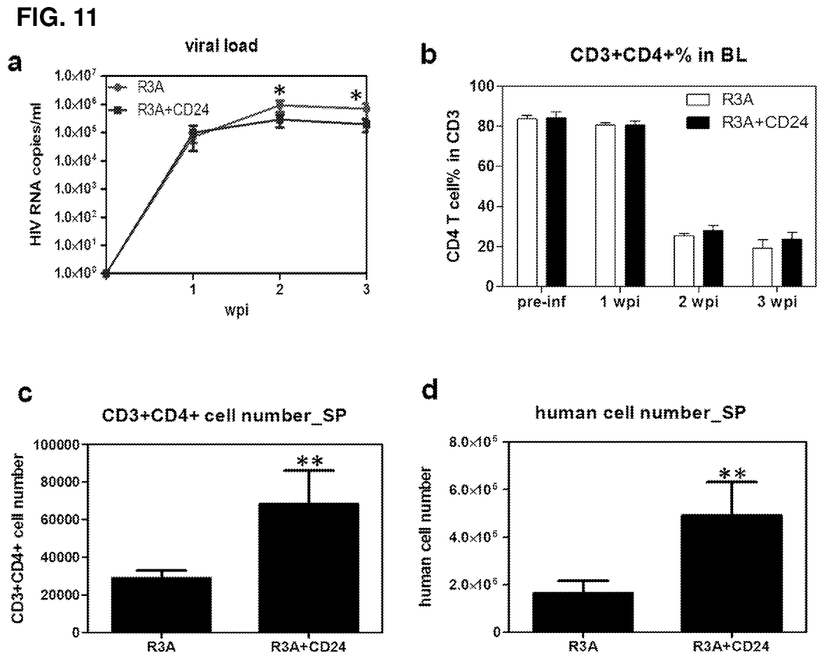

CD24Fc treatment reduces HIV-1 viral load and protects CD4+ T cells from depletion in the spleen of mice with acute HIV infection. It was first investigated whether CD24Fc treatment influences HIV-1 replication and immune-pathogenesis in acute HIV-1 infection with humanized mice. As shown in FIG. 11A, in the vehicle-treated group, R3A replication was rapidly increased to 1×10⁶ copies/ml at 1 week post-infection (wpi), then it gradually increased to 10⁸ copies/ml at 2-3 wpi. In the CD24Fc treated group, R3A increase in the first week was unaffected. However, no further increase was observed at 2 and 3 wpi. Nevertheless, CD24Fc treatment did not abort reduction of CD4 T cell frequency among CD3⁺ T cells (FIG. 11B). Notably, CD24Fc treatment significantly increased the numbers of CD4⁺ T cells in the spleen at the termination of the mice at 3 wpi (FIG. 11C). This increase of CD4⁺ T cell number corresponded with the increase of the total human lymphocytes in the spleen of humanized mice (FIG. 11D). These data indicated that CD24Fc has the potential to reduce HIV-1 viral load and protect CD4⁺ T cells from depletion in the spleen of humanized mice with acute HIV infection.

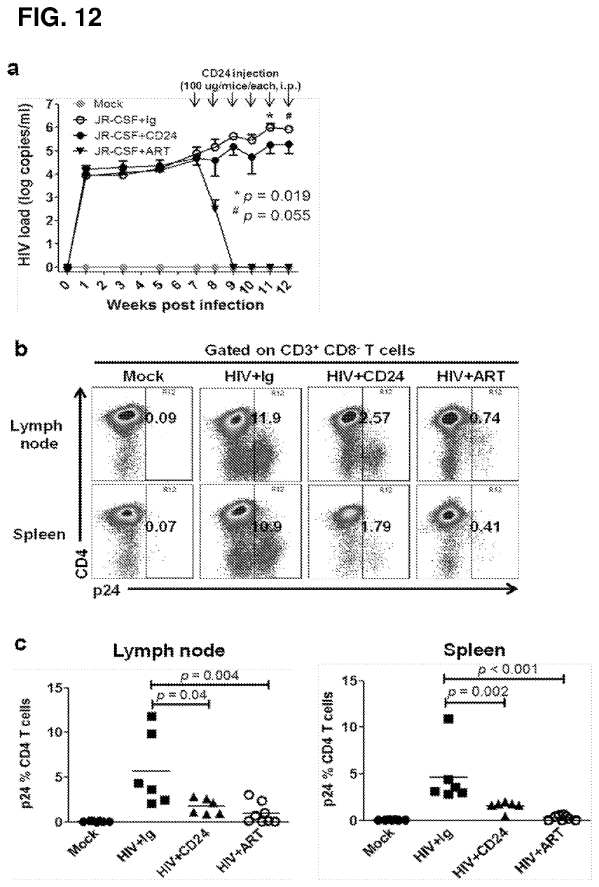

CD24Fc Treatment Reduced HIV-1 Replication in Humanized Mice with Chronic HIV Infection. Next, it was investigated whether CD24Fc treatment influences chronic HIV-1 replication in humanized mice after JR-CSF infection. Plasma HIV-1 load in these mice was serially detected, and it was found that HIV-1 load was persistently increased in plasma since the onset of HIV-1 infection. As expected, combined antiretroviral therapy (cART) completely inhibited plasma HIV-1 load to undetectable levels. CD24Fc treatment was able to limit the increase of plasma HIV-1 load, thus leading to significantly lower levels of HIV-1 load in treated mice compared to HIV-1 infected mice (FIG. 12A). When the mice were terminated, p24 expression by CD4+ T cells was further detected in various lymph tissues (FIG. 12B). It was observed that CD24Fc treatment reduced the percentage of p24-expressing cells by more than 5-fold. Again, as expected, cART appeared even more effective, causing a 10- to 20-fold reduction (FIG. 12B). Combined data from studies involving 6-7 mice per group further confirmed the significant reduction in p24-expressing cells in the spleen and lymph node (FIG. 12C). These data indicated that CD24Fc treatment suppresses chronic HIV-1 replication in humanized mice.

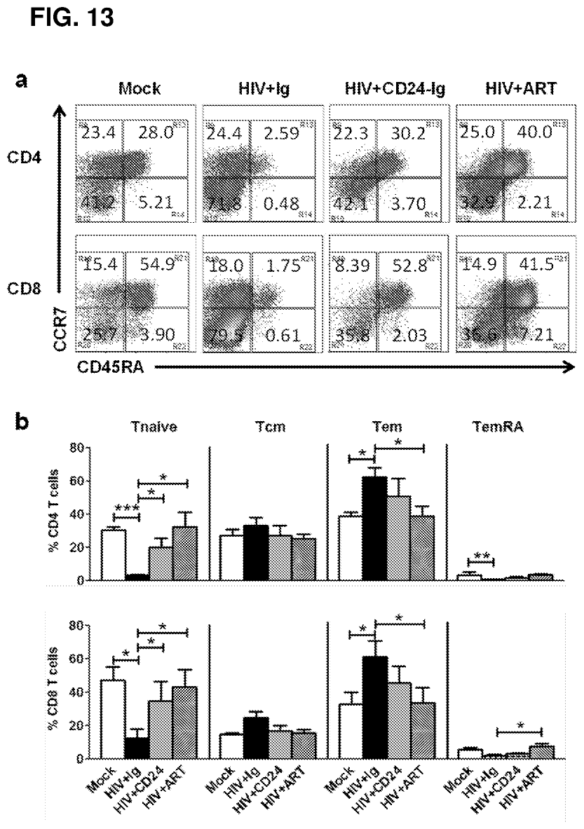

CD24Fc Treatment Replenished the Naïve T-Cell Compartment in Humanized Mice with Chronic HIV-1 Infection. The effects of CD24Fc treatment on CD4 T cell subsets were tested in humanized mice with HIV-1 infection using the CCR7 and CD45RA as markers for naïve T cells (FIG. 13A). In control Ig-treated mice, HIV-1 infection significantly decreased the proportion of CD45RA⁺CCR7⁺ naïve T cells and increased the proportion of CD45RA-CCR7− effector memory T cell subsets in both CD4 and CD8 T cells. Importantly, CD24Fc treatment markedly reversed the skew of CD4 and CD8 T cell subsets in spleen of humanized mice with HIV-1 infection. Remarkably, CD24Fc was almost as effective as the cART in preventing pathogenic loss of naïve T cells in chronically infected mice (FIG. 13B).

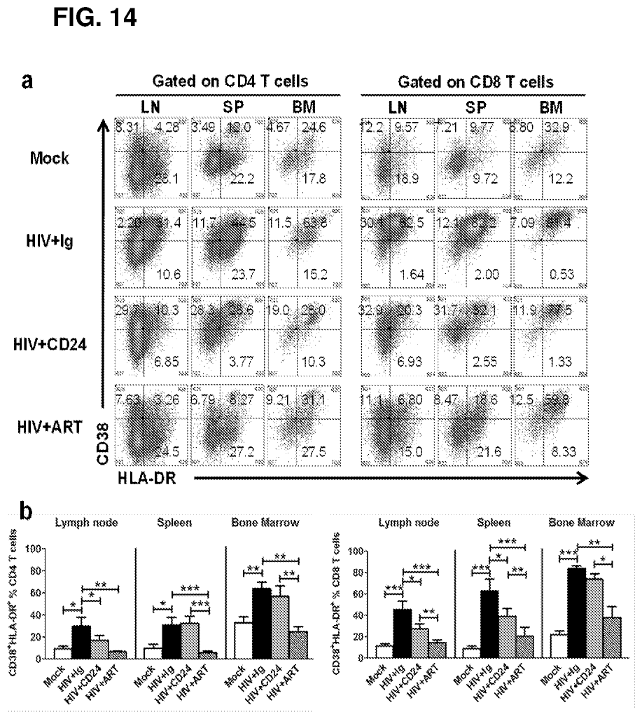

CD24Fc Treatment Reduced Immune Over-Activation in vivo in Humanized Mice with Chronic HIV-1 Infection. It was further investigated whether CD24Fc treatment has the potential to rescue HIV-1-induced immune pathogenesis. Immune over-activation has been demonstrated to be a hall mark of disease progression in human with chronic HIV-1 infection. Therefore, the activation of CD4 and CD8 T cells was detected in various lymphoid tissues (FIG. 14A). Similar to HIV-1-infected patients, HIV-1 infection significantly increased the proportion of CD38+HLA-DR+ CD4+ T cells and CD8+ T cells in lymph node, spleen and bone marrow of humanized mice compared to mock mice. cART largely reduced the activation of CD4+ and CD8+ T cells to nearly normal levels in all of lymphoid tissues tested in these mice with HIV-1 infection, as expected. Importantly, CD24Fc treatment also significantly reduced the activation of CD4+ T cells in lymphoid node and activated CD8+ T cells from the lymph node and spleen during HIV-1 infection (FIG. 14B). These data show that CD24Fc has the potential to restrict the immune over-activation and inflammation induced by persistent HIV-1 infection.

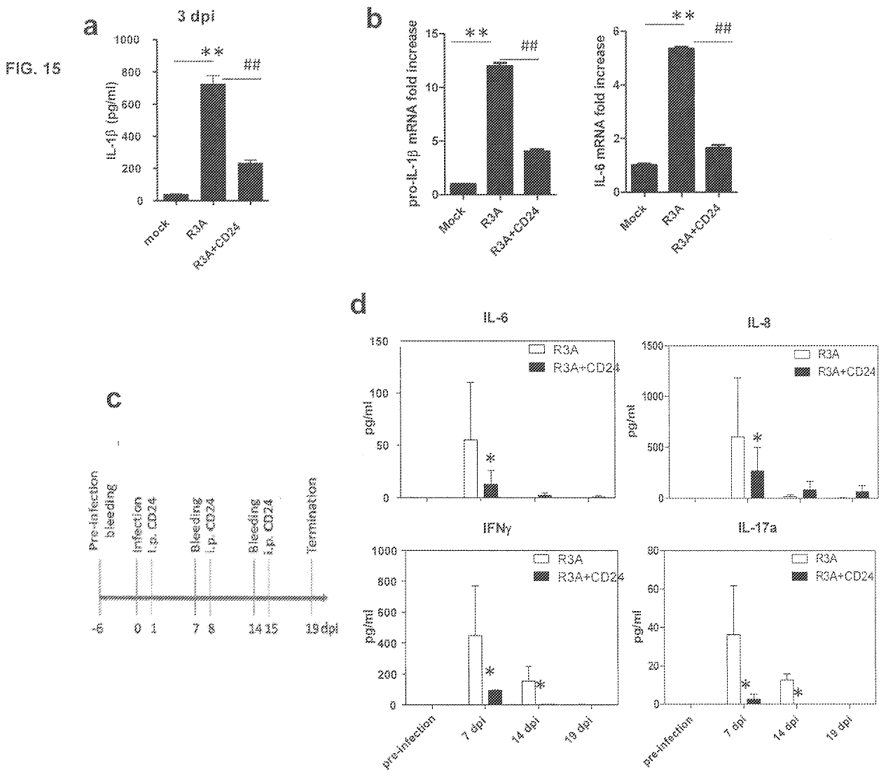

CD24Fc Treatment Blocks HIV-1 Induced Pro-Inflammatory Cytokine Production in vitro and in vivo. It was tested whether CD24Fc can reduce pro-inflammatory cytokines. As shown in FIG. 15A, in vitro, R3A infection induced pro-inflammatory cytokine IL-1β protein, and this induction was largely abrogated by CD24Fc (FIG. 15A). Likewise, CD24Fc also significantly reduced the IL6 and Pro-IL-1β mRNA (FIG. 15B). The effects of CD24Fc treatment on T cell activation and pro-inflammatory cytokines in acute HIV-1 infection were further tested, as diagrammed in FIG. 15C. Importantly, CD24Fc treatment significantly inhibited plasma IL-6, IL-8, IFN-γ and IL-17a (FIG. 15D).

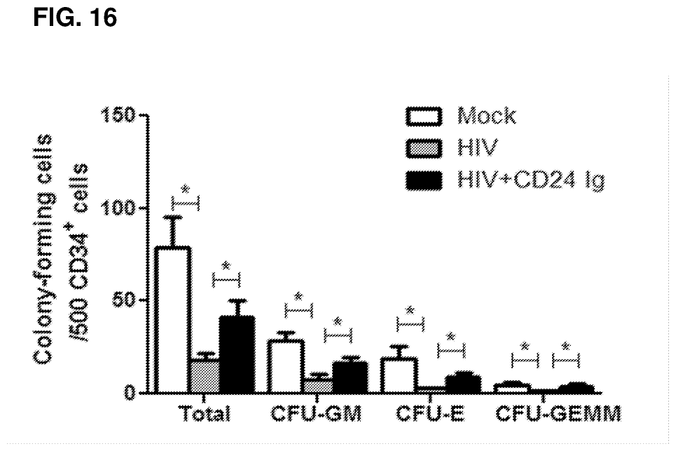

CD24Fc Treatment Rescues Hematopoietic Suppression Induced by Persistent HIV-1 Infection. Finally, the effects of CD24Fc treatment on BM hematopoietic suppression during HIV-1 infection were evaluated. Lin-CD34+ cells were purified for colony-forming assays, including granulocyte/macrophage (GM), erythroid (E) and granulocyte/erythroid/macrophage/megakaryocyte (GEMM) subsets. The results demonstrate that CD24Fc treatment also significantly enhances CFU activity of the total population as well as each colony type individually, as compared with HIV-1 infection alone (FIG. 16).

### Example 6

### CD24 Pharmacokinetics in Humans

This example shows an analysis of the pharmacokinetics of a CD24 protein in humans. This was derived from a Phase I, randomized, double-blind, placebo-controlled, single ascending dose study to assess the safety, tolerability, and PK of CD24Fc in healthy male and female adult subjects. A total of 40 subjects in 5 cohorts of 8 subjects each were enrolled in this study. Six of the 8 subjects in each cohort received study drug and 2 subjects received placebo (0.9% sodium chloride, saline). The first cohort was dosed with 10 mg. Succeeding cohorts received 30 mg, 60 mg, 120 mg, and 240 mg of CD24Fc or matching placebo and were dosed at least 3 weeks apart to allow for review of safety and tolerability data for each prior cohort. Administration of the next higher dose to a new cohort of subjects was permitted only if adequate safety and tolerability had been demonstrated.

In each cohort, the initial 2 subjects were 1 study drug recipient and 1 placebo recipient on Day 1. The 3rd to 5th and 6th to 8th subjects were dosed after Day 7 (a minimum of 24 hours apart between the subgroups). Each subject was dosed at least 1 hour apart in the same subgroup. If necessary, dosing of the rest of subjects was delayed pending review of any significant safety issues that may have arisen during the post-dose period involving the first or second subgroups in that cohort. The subsequent cohort was dosed at least 3 weeks after the prior cohort.

Screening Period:

The Screening Visit (Visit 1) occurred up to 21 days prior to the beginning of the active treatment period. After providing informed consent, subjects underwent screening procedures for eligibility.

Treatment Period:

Subjects were admitted to the Clinical Pharmacology Unit (CPU) on Day −1 (Visit 2), and the randomized treatment period began on Day 1 following a 10-hour minimum overnight fast. Subjects were randomly assigned to treatment with CD24Fc or placebo as a single dose. Subjects remained confined until the morning of Day 4.

Follow-Up:

All subjects returned to the CPU on Day 7, Day 14, Day 21, Day 28, and Day 42 (±1 day) for follow-up visits (Visit 3, Visit 4, Visit 5, Visit 6, and Visit 7). Visit 7 was the final visit for all subjects.

Duration of Treatment: The total study duration for each subject was up to 63 days. Single-dose administration occurred on Day 1.

Number of Subjects:

Planned: 40 subjects

Screened: 224 subjects

Randomized: 40 subjects

Completed: 39 subjects

Discontinued: 1 subject

Diagnosis and Main Criteria for Inclusion: The population for this study was healthy males and females between the ages of 18 and 55 years, inclusive, with a body mass index between 18 kg/m² and 30 kg/m², inclusive.

Investigational Product and Comparator Information:

CD24Fc: single dose of 10 mg, 30 mg, 60 mg, 120 mg, or 240 mg administered via IV infusion; lot number: 09MM-036. CD24Fc was a fully humanized fusion protein consisting of the mature sequence of human CD24 and the fragment crystallizable region of human immunoglobulin G1 (IgG1Fc). CD24Fc was supplied as a sterile, clear, colorless, preservative-free, aqueous solution for IV administration. CD24Fc was formulated as single dose injection solution, at a concentration of 10 mg/mL and a pH of 7.2. Each CD24Fc vial contained 160 mg of CD24Fc, 5.3 mg of sodium chloride, 32.6 mg of sodium phosphate dibasic heptahydrate, and 140 mg of sodium phosphate monobasic monohydrate in 16 mL 0.2 mL of CD24Fc. CD24Fc was supplied in clear borosilicate glass vials with chlorobutyl rubber stoppers and aluminum flip-off seals.

Matching placebo (0.9% sodium chloride, saline) administered via IV infusion; lot numbers: P296855, P311852, P300715, P315952.

The intent-to-treat (ITT) Population consisted of all subjects who received at least 1 dose of the study drug. The ITT Population was the primary analysis population for subject information and safety evaluation.

Clinical laboratory evaluations (chemistry, hematology, and urinalysis) were summarized by treatment and visit. Change from baseline was also summarized. Vital signs (blood pressure, heart rate, respiratory rate, and temperature) were summarized by treatment and time point. Change from baseline was also summarized. All physical examination data were listed. Electrocardiogram parameters and the change from baseline were summarized. Overall interpretations were listed.

Plasma CD24Fc Concentration

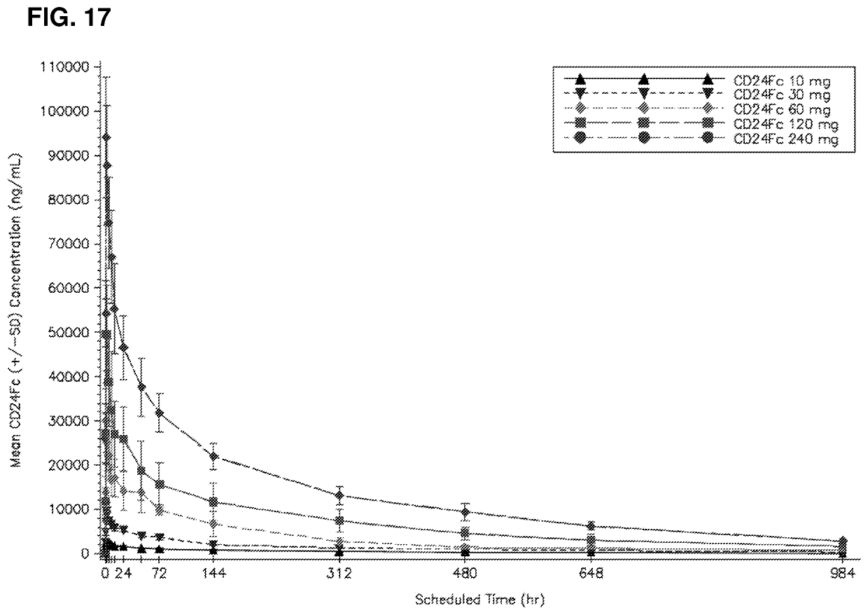

As shown in FIG. 17, the mean plasma concentration of CD24Fc increased proportionally to the dose of CD24Fc administered. For all dose groups except 120 mg, the maximum mean plasma concentration of CD24Fc was reached at 1 hour post-dose. The maximum mean plasma concentration of CD24Fc for the 120 mg group was reached at 2 hours post-dose. By Day 42 (984 hours), the mean plasma concentration of CD24Fc for all groups had decreased to between 2% and 4% of the maximum mean plasma concentration.

Table 1 summarizes the plasma CD24Fc PK parameters by treatment for the PK Evaluable Population.

TABLE 1
Summary of Plasma CD24Fc Pharmacokinetic Parameters by Treatment - PK Evaluable Population

| | CD24Fc | | CD24Fc | | CD24Fc | | CD24Fc | | CD24Fc | |
|---|---|---|---|---|---|---|---|---|---|---|
| Parameter | 10 mg | | 30 mg | | 60 mg | | 120 mg | | 240 mg | |
| Statistic | (N = 6) | | (N = 6) | | (N = 6) | | (N = 6) | | (N = 6) | |
| Cₘₐₓ (ng/mL) | | | | | | | | | | |
| n | 6 | | 6 | | 6 | | 6 | | 6 | |
| Mean (SD) | 2495 | (576) | 9735 | (1715) | 30 083 | (7179) | 52 435 | (9910) | 95 865 | (10734) |
| CV % | 23.1 | | 17.6 | | 23.9 | | 18.9 | | 11.2 | |
| Median | 2371 | | 9218 | | 29 026 | | 50 401 | | 93 206 | |
| Min, Max | 1,967, | | 8,583, | | 22,557, | | 40,434, | | 81,296, | |
| | 3,390 | | 13,086 | | 42,628 | | 65,704 | | 110,110 | |
| Geometric | 2,442 | | 9,625 | | 29,424 | | 51,666 | | 95,365 | |
| mean | | | | | | | | | | |
| Geometric | 22.8 | | 16.1 | | 23.0 | | 19.0 | | 11.2 | |
| CV % | | | | | | | | | | |
| AUC_{0-42 d} | | | | | | | | | | |
| (ng*hr/mL) | | | | | | | | | | |
| n | 6 | | 6 | | 6 | | 6 | | 6 | |
| Mean (SD) | 423,061 | (99,615) | 1,282,430 | (88,798) | 3,226,255 | (702,862) | 6,541,501 | (2,190,944) | 12,704,705 | (1,918,596) |
| CV % | 23.5 | | 6.9 | | 21.8 | | 33.5 | | 15.1 | |
| Median | 434,043 | | 1,302,719 | | 3,124,933 | | 5,785,142 | | 12,563,426 | |
| Min, Max | 291,020, | | 1,175,733, | | 2,487,550, | | 4,485,193, | | 10,466,635, | |
| | 528,079 | | 1,403,024 | | 4,139,748 | | 9,415,266 | | 15,693,606 | |
| Geometric | 412,795 | | 1,279,851 | | 3,163,252 | | 6,249,552 | | 12,586,731 | |
| mean | | | | | | | | | | |
| Geometric | 25.0 | | 7.0 | | 22.0 | | 33.8 | | 15.0 | |
| CV % | | | | | | | | | | |
| AUC_{0-inf} | | | | | | | | | | |
| (ng*hr/mL) | | | | | | | | | | |
| n | 6 | | 6 | | 6 | | 6 | | 6 | |
| Mean (SD) | 462,260 | (116,040) | 1,434,464 | (131,316) | 3,497,196 | (705,653) | 7,198,196 | (2,458,320) | 13,861,796 | (1,962,780) |
| CV % | 25.1 | | 9.2 | | 20.2 | | 34.2 | | 14.2 | |
| Median | 470,426 | | 1,422,205 | | 3,519,732 | | 6,463,665 | | 13,713,034 | |
| Min, Max | 310,956, | | 1,281,715, | | 2,703,655, | | 4,910,640, | | 11,822,988, | |
| | 596,599 | | 1,650,503 | | 4,309,023 | | 10,479,940 | | 17,175,236 | |
| Geometric | 449,583 | | 1,429,578 | | 3,437,036 | | 6,862,129 | | 13,750,972 | |
| mean | | | | | | | | | | |
| Geometric | 26.7 | | 9.0 | | 20.7 | | 34.6 | | 13.8 | |
| CV % | | | | | | | | | | |
| Tₘₐₓ (hr) | | | | | | | | | | |
| n | 6 | | 6 | | 6 | | 6 | | 6 | |
| Mean (SD) | 1.15 | (0.42) | 1.17 | (0.41) | 1.01 | (0.01) | 1.34 | (0.51) | 1.33 | (0.52) |
| CV % | 36.1 | | 35.0 | | 1.2 | | 38.0 | | 38.7 | |
| Median | 1.00 | | 1.00 | | 1.00 | | 1.03 | | 1.00 | |
| Min, Max | 0.92, | | 1.00, | | 1.00, | | 1.00, | | 1.00, | |
| | 2.00 | | 2.00 | | 1.03 | | 2.00 | | 2.00 | |
| t½ (hr) | | | | | | | | | | |
| n | 6 | | 6 | | 6 | | 6 | | 6 | |
| Mean (SD) | 280.83 | (22.37) | 327.10 | (41.32) | 279.82 | (65.59) | 286.45 | (23.38) | 285.33 | (24.33) |
| CV % | 8.0 | | 12.6 | | 23.4 | | 8.2 | | 8.5 | |
| Median | 279.61 | | 317.23 | | 264.69 | | 290.76 | | 287.74 | |
| Min, Max | 258.87, | | 289.82, | | 210.18, | | 243.89, | | 249.24, | |
| | 321.26 | | 394.24 | | 362.46 | | 309.26 | | 322.26 | |
| AUCextr (%) | | | | | | | | | | |
| n | 6 | | 6 | | 6 | | 6 | | 6 | |
| Mean (SD) | 7.61 | (2.14) | 10.44 | (2.94) | 7.88 | (4.26) | 8.92 | (1.94) | 8.46 | (1.99) |
| CV % | 28.1 | | 28.2 | | 54.0 | | 21.8 | | 23.5 | |
| Median | 7.16 | | 10.01 | | 6.35 | | 9.27 | | 8.45 | |
| Min, Max | 5.46, | | 7.10, | | 3.92, | | 5.49, | | 5.56, | |
| | 11.47 | | 15.05 | | 14.48 | | 10.99 | | 11.50 | |
| CL (L/hr) | | | | | | | | | | |
| n | 6 | | 6 | | 6 | | 6 | | 6 | |
| Mean (SD) | 0.0229 | (0.0061) | 0.0211 | (0.0019) | 0.0178 | (0.0036) | 0.0183 | (0.0058) | 0.0176 | (0.0023) |
| CV % | 26.7 | | 8.8 | | 20.5 | | 31.7 | | 13.3 | |
| Median | 0.0216 | | 0.0211 | | 0.0173 | | 0.0191 | | 0.0175 | |
| Min, Max | 0.0168, | | 0.0182, | | 0.0139, | | 0.0115, | | 0.0140, | |
| | 0.0322 | | 0.0234 | | 0.0222 | | 0.0244 | | 0.0203 | |
| Vd (L) | | | | | | | | | | |
| n | 6 | | 6 | | 6 | | 6 | | 6 | |
| Mean (SD) | 9.153 | (1.943) | 9.867 | (0.804) | 7.289 | (2.592) | 7.491 | (2.202) | 7.276 | (1.426) |

| CV % | 21.2 | 8.1 | 35.6 | 29.4 | 19.6 |
|---|---|---|---|---|---|
| Median | 8.507 | 10.007 | 7.486 | 7.691 | 7.151 |
| Min, Max | 7.326, | 8.771, | 4.222, | 4.933, | 5.814, |
| | 12.010 | 10.958 | 11.139 | 9.974 | 9.438 |

AUC_{0-42 d} = area under the concentration-time curve from time 0 to 42 days; AUC_{0-inf} = area under the concentration-time curve extrapolated from time 0 to infinity; AUCₑₓₜᵣ = percentage of AUC_{0-inf} that was due to extrapolation from the time of the last measurable concentration, per subject, to infinity; CL = total body clearance; Cₘₐₓ = maximum observed plasma drug concentration; CV % = coefficient of variation; Min = minimum; Max = maximum; SD = standard deviation; t_{1/2} = terminal elimination half-life; Tₘₐₓ = time of maximum observed plasma drug concentration; V_{d} = volume of distribution.

Plasma CD24Fc Dose Proportionality Analysis

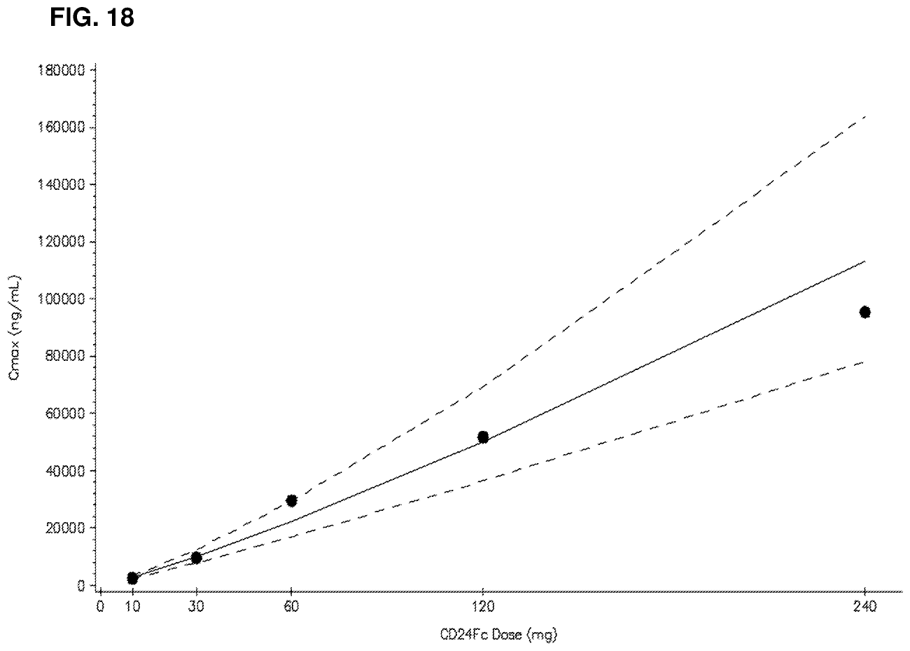

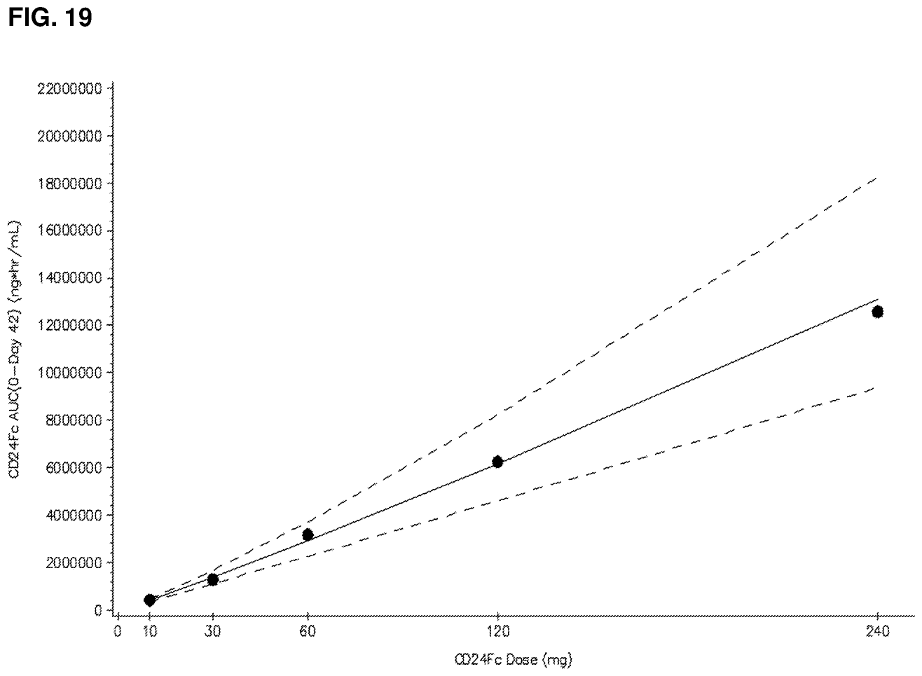

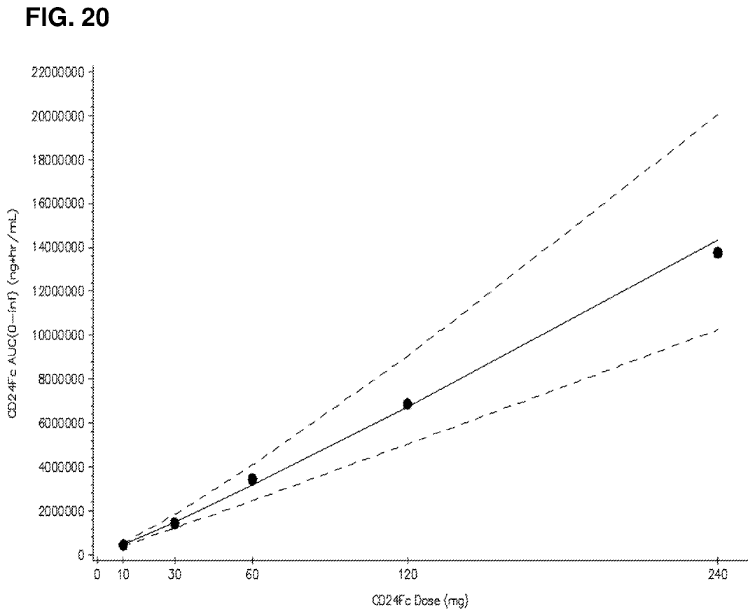

FIG. 18 shows a dose proportionality plot of CD24Fc Cₘₐₓ versus dose for the PK Evaluable Population. FIG. 19 shows a dose proportionality plot of CD24Fc AUC_{0-42d} versus dose for the PK Evaluable Population. FIG. 20 shows a dose proportionality plot of CD24Fc AUC_{0-inf} versus dose for the PK Evaluable Population. Table 2 shows a power analysis of dose proportionality.

TABLE 2
Power Analysis of Dose Proportionality: Plasma CD24Fc Pharmacokinetic Parameters - PK Evaluable Population

| | CD24Fc | CD24Fc | CD24Fc | CD24Fc | CD24Fc | Dose Proportionality | | |
|---|---|---|---|---|---|---|---|---|
| Parameter | 10 mg | 30 mg | 60 mg | 120 mg | 240 mg | Slope | Standard | |
| Statistic | (N = 6) | (N = 6) | (N = 6) | (N = 6) | (N = 6) | Estimate | Error | 90% CI |
| Cₘₐₓ (ng/mL) | | | | | | 1.172 | 0.040 | (1.105, |
| | | | | | | | | 1.240) |
| Geometric | 2,441.8 | 9,624.9 | 29,424.4 | 51,666.4 | 95,364.9 | | | |
| mean | | | | | | | | |
| Geometric | 22.8 | 16.1 | 23.0 | 19.0 | 11.2 | | | |
| CV % | | | | | | | | |
| AUC_{0-42 d} | | | | | | 1.088 | 0.036 | (1.027, |
| (ng*hr/mL) | | | | | | | | 1.148) |
| Geometric | 412,794.8 | 1,279,850.8 | 3,163,251.7 | 6,249,551.9 | 12,586,731.3 | | | |
| mean | | | | | | | | |
| Geometric | 25.0 | 7.0 | 22.0 | 33.8 | 15.0 | | | |
| CV % | | | | | | | | |
| AUC_{0-inf} | | | | | | 1.087 | 0.036 | (1.026, |
| (ng*hr/mL) | | | | | | | | 1.148) |
| Geometric | 449,583.5 | 1,429,577.5 | 3,437,035.6 | 6,862,128.7 | 13,750,972.4 | | | |
| mean | | | | | | | | |
| Geometric | 26.7 | 9.0 | 20.7 | 34.6 | 13.8 | | | |
| CV % | | | | | | | | |

Geometric CV % = 100*sqrt(exp(SD²) − 1), where SD was the standard deviation of the log-transformed data. The power model was fitted by restricted maximum likelihood, regressing the log-transformed PK parameter on log transformed dose. Both the intercept and slope were fitted as fixed effects. Dose proportionality was not rejected if the 90% CI lies within (0.8, 1.25).
AUC_{0-42 d} = area under the concentration-time curve from time 0 to 42 days; AUC₀₋ᵢₙₜ = area under the concentration-time curve extrapolated from time 0 to infinity; CI = confidence interval; Cₘₐₓ = maximum observed plasma drug concentration; CV % = coefficient of variation; PK = pharmacokinetic; SD = standard deviation.

The Cₘₐₓ slope estimate was 1.172 with a 90% CI of 1.105 to 1.240. The AUC_{0-42d} slope estimate was 1.088 with a 90% CI of 1.027 to 1.148. The AUC_{0-inf} slope estimate was 1.087 with a 90% CI of 1.026 to 1.1.

Pharmacokinetic Conclusions

The Cₘₐₓ and AUCs of plasma CD24Fc increased proportionally to the doses administered in mouse, monkey and human. The plasma CD24Fc reached Tₘₐₓ between 1.01 and 1.34 hours. The t_{1/2} of plasma CD24Fc ranged between 280.83 and 327.10 hours.

### Example 7

### CD24 can be Used to Treat Graft Versus Host Disease

This Example demonstrates that CD24 can treat or prevent GvHD by negatively regulating host response to cellular DAMPs, without affecting the graft versus host (GVL) effects of the transplanted cells. NK cells can enhance engraftment and mediate graft-versus-leukemia following allogeneic HSCT, but the potency of graft-versus-leukemia mediated by naturally reconstituting NK cells following HSCT is limited. Preclinical studies demonstrate that activation of NK cells upregulates activating receptor expression and augments killing capacity (Shah et al 2015). This was then tested in a clinical trial studying the adoptive transfer of donor-derived activated NK cells (aNK-DLI) following HLA-matched, T-cell—depleted nonmyeloablative peripheral blood stem cell transplantation in children and young adults with ultra-high-risk solid tumors. aNK-DLI demonstrated potent killing capacity and displayed high levels of activating receptor expression. However, 5 of 9 transplant recipients experienced acute graft-versus-host disease (GVHD) following aNK-DLI, with grade 4 GVHD observed in 3 subjects. GVHD was more common in matched unrelated donor vs matched sibling donor recipients and was associated with higher donor CD3 chimerism. Given that the T-cell dose was below the threshold required for GVHD in this setting, it was concluded that aNK-DLI contributed to the acute GVHD observed, likely by augmenting underlying T-cell alloreactivity. Accordingly, the CD24 proteins described herein can be used to treat or provent GvHD in an animal.

### REFERENCES

1. Munoz L E, Janko C, Schulze C, Schorn C, Sarter K, Schett G, Herrmann M. Autoimmunity and chronic inflammation—two clearance-related steps in the etiopathogenesis of SLE. Autoimmun Rev. 2010; 10(1):38-42. Epub 2010/09/08. doi: 10.1016/j.autrev.2010.08.015. PubMed PMID: 20817127.
2. Urbonaviciute V, Furnrohr B G, Meister S, Munoz L, Heyder P, De Marchis F, Bianchi M E, Kirschning C, Wagner H, Manfredi A A, Kalden J R, Schett G, Rovere-Querini P, Herrmann M, Voll R E. Induction of inflammatory and immune responses by HMGB1-nucleosome complexes: implications for the pathogenesis of SLE. J Exp Med. 2008; 205(13):3007-18. PubMed PMID: 19064698.
3. Wen Z, Xu L, Chen X, Xu W, Yin Z, Gao X, Xiong S. Autoantibody induction by DNA-containing immune complexes requires HMGB1 with the TLR2/microRNA-155 pathway. Journal of Immunology. 2013; 190(11):5411-22. Epub 2013/04/26. doi: 10.4049/jimmunol.1203301. PubMed PMID: 23616573.
4. Andersson U, Harris H E. The role of HMGB1 in the pathogenesis of rheumatic disease. Biochim Biophys Acta. 1799(1-2):141-8. PubMed PMID: 20123076.
5. Ostberg T, Kawane K, Nagata S, Yang H, Chavan S, Klevenvall L, Bianchi M, Harris H E, Andersson U, Palmblad K. Protective targeting of HMGB1 in a spontaneous arthritis model. Arthritis Rheum. 2010; 62:2963-72. PubMed PMID: 20533288.
6. Rice J W, Veal J M, Fadden R P, Barabasz A F, Partridge J M, Barta T E, Dubois L G, Huang K H, Mabbett S R, Silinski M A, Steed P M, Hall S E. Small molecule inhibitors of Hsp90 potently affect inflammatory disease pathways and exhibit activity in models of rheumatoid arthritis. Arthritis Rheum. 2008; 58(12):3765-75. PubMed PMID: 19035474.
7. Ahrens S, Zelenay S, Sancho D, Hanc P, Kjaer S, Feest C, Fletcher G, Durkin C, Postigo A, Skehel M, Batista F, Thompson B, Way M, Reis e Sousa C, Schulz O. F-actin is an evolutionarily conserved damage-associated molecular pattern recognized by DNGR-1, a receptor for dead cells. Immunity. 2012; 36(4):635-45. Epub 2012/04/10. doi: S1074-7613(12)00126-4 [pii] 10.1016/j.immuni.2012.03.008. PubMed PMID: 22483800.
8. Yamasaki S, Ishikawa E, Sakuma M, Hara H, Ogata K, Saito T. Mincle is an ITAM-coupled activating receptor that senses damaged cells. Nat Immunol. 2008; 9(10):1179-88. Epub 2008 Sep. 9. doi: ni.1651 [pii] 10.1038/ni.1651. PubMed PMID: 18776906.
9. Cavassani K A, Ishii M, Wen H, Schaller M A, Lincoln P M, Lukacs N W, Hogaboam C M, Kunkel S L. TLR3 is an endogenous sensor of tissue necrosis during acute inflammatory events. Journal of Experimental Medicine. 2008; 205(11):2609-21. PubMed PMID: 18838547.
10. Ivanov S, Dragoi A M, Wang X, Dallacosta C, Louten J, Musco G, Sitia G, Yap G S, Wan Y, Biron C A, Bianchi M E, Wang H, Chu W M. A novel role for HMGB1 in TLR9-mediated inflammatory responses to CpG-DNA. Blood. 2007; 110(6):1970-81. Epub 2007/06/06. doi: blood-2006-09-044776 [pii] 10.1182/blood-2006-09-044776. PubMed PMID: 17548579; PMCID: 1976374.
11. Sims G P, Rowe D C, Rietdijk S T, Herbst R, Coyle A J. HMGB1 and RAGE in inflammation and cancer. Annu Rev Immunol. 28:367-88. PubMed PMID: 20192808.
12. van Beijnum J R, Buurman W A, Griffioen A W. Convergence and amplification of toll-like receptor (TLR) and receptor for advanced glycation end products (RAGE) signaling pathways via high mobility group B1 (HMGB1). Angiogenesis. 2008; 11(1):91-9. PubMed PMID: 18264787.
13. Warger T, Hilf N, Rechtsteiner G, Haselmayer P, Carrick D M, Jonuleit H, von Landenberg P, Rammensee H G, Nicchitta C V, Radsak M P, Schild H. Interaction of TLR2 and TLR4 ligands with the N-terminal domain of Gp96 amplifies innate and adaptive immune responses. The Journal of biological chemistry. 2006; 281(32):22545-53. PubMed PMID: 16754684.
14. Zhang Q, Raoof M, Chen Y, Sumi Y, Sursal T, Junger W, Brohi K, Itagaki K, Hauser C J. Circulating mitochondrial DAMPs cause inflammatory responses to injury. Nature. 2010; 464(7285):104-7. Epub 2010/03/06. doi: nature 08780 [pii] 10.1038/nature08780. PubMed PMID: 20203610; PMCID: 2843437.
15. Chen G Y, Tang J, Zheng P, Liu Y. CD24 and Siglec-10 selectively repress tissue damage-induced immune responses. Science. 2009; 323(5922):1722-5. doi: 10.1126/science.1168988. PubMed PMID: 19264983; PMCID: PMC2765686.
16. Chen G Y, Chen X, King S, Cavassani K A, Cheng J, Zheng X, Cao H, Yu H, Qu J, Fang D, Wu W, Bai X F, Liu J Q, Woodiga S A, Chen C, Sun L, Hogaboam C M, Kunkel S L, Zheng P, Liu Y. Amelioration of sepsis by inhibiting sialidase-mediated disruption of the CD24-SiglecG interaction. Nat Biotechnol. 2011; 29(5):428-35. doi: 10.1038/nbt.1846. PubMed PMID: 21478876; PMCID: PMC4090080.
17. Chen W, Han C, Xie B, Hu X, Yu Q, Shi L, Wang Q, Li D, Wang J, Zheng P, Liu Y, Cao X. Induction of Siglec-G by RNA viruses inhibits the innate immune response by promoting RIG-I degradation. Cell. 2013; 152(3):467-78. Epub 2013/02/05. doi: 10.1016/j.cell.2013.01.011. PubMed PMID: 23374343.
18. Goris A, Maranian M, Walton A, Yeo T W, Ban M, Gray J, Dubois B, Compston A, Sawcer S. CD24 Ala/Val polymorphism and multiple sclerosis. Journal of neuroimmunology. 2006. PubMed PMID: 16631259.
19. Otaegui D, Saenz A, Camano P, Blazquez L, Goicoechea M, Ruiz-Martinez J, Olaskoaga J, Emparanza J A, Lopez de Munain A. CD24 V/V is an allele associated with the risk of developing multiple sclerosis in the Spanish population. Multiple sclerosis (Houndmills, Basingstoke, England). 2006; 12(4):511-4. Epub 2006/08/12. PubMed PMID: 16900767.
20. Wang L, Lin S, Rammohan K, Liu Z, Liu J, Liu R-H, Guinther N, Zhou Q, Wang T, Zheng X, Birmingham D J, Rovin B H, Herbert L A, Wu Y, Lynn D J, Cooke G, Yu C Y, Zheng P, Liu Y. A di-nucleotide deletion in CD24 confers protection against autoimmune diseases. Plos Genetics. 2007; 3:e49.
21. Zhou Q, Rammohan K, Lin S, Robinson N, Li O, Liu X, Bai X F, Yin L, Scarberry B, Du P, You M, Guan K, Zheng P, Liu Y. CD24 is a genetic modifier for risk and progression of multiple sclerosis. Proc Natl Acad Sci USA. 2003; 100(25):15041-6. doi: 10.1073/pnas.2533866100. PubMed PMID: 14657362; PMCID: PMC299898.
22. Sanchez E, Abelson A K, Sabio J M, Gonzalez-Gay M A, Ortego-Centeno N, Jimenez-Alonso J, de Ramon E, Sanchez-Roman J, Lopez-Nevot M A, Gunnarsson I, Svenungsson E, Sturfelt G, Truedsson L, Jonsen A, Gonzalez-Escribano M F, Witte T, Alarcon-Riquelme M E, Martin J. Association of a CD24 gene polymorphism with susceptibility to systemic lupus erythematosus. Arthritis Rheum. 2007; 56(9):3080-6. Epub 2007/09/01. doi: 10.1002/art.22871. PubMed PMID: 17763438.
23. Sanchez E, Fernandez-Gutierrez B, Gonzalez-Gay M A, Balsa A, Garcia A, Rodriguez L, Pascual-Salcedo D, Gonzalez-Escribano M F, Martin J. Investigating the role of CD24 gene polymorphisms in rheumatoid arthritis. Annals of the rheumatic diseases. 2008; 67(8):1197-8. Epub 2008/07/16. doi: 10.1136/ard.2007.084475. PubMed PMID: 18621973.
24. Rueda B, Miranda-Filloy J A, Martin J, Gonzalez-Gay M A. Association of CD24 gene polymorphisms with susceptibility to biopsy-proven giant cell arteritis. The Journal of rheumatology. 2008; 35(5):850-4. Epub 2008/04/03. PubMed PMID: 18381780.
25. Lee Y H, Bae S C. Association between functional CD24 polymorphisms and susceptibility to autoimmune diseases: A meta-analysis. Cell Mol Biol (Noisy-le-grand). 2015; 61(8):97-104. Epub 2016/01/01. PubMed PMID: 26718436.
26. Bokers S, Urbat A, Daniel C, Amann K, Smith K G, Espeli M, Nitschke L. Siglec-G deficiency leads to more severe collagen-induced arthritis and earlier onset of lupus-like symptoms in MRL/lpr mice. Journal of immunology (Baltimore, Md.: 1950). 2014; 192(7):2994-3002. Epub 2014/03/07. doi: 10.4049/jimmunol.1303367. PubMed PMID: 24600033.
27. Wigren M, Nilsson J, Kaplan M J. Pathogenic immunity in systemic lupus erythematosus and atherosclerosis: common mechanisms and possible targets for intervention. Journal of internal medicine. 2015; 278(5):494-506. Epub 2015/02/28. doi: 10.1111/joim.12357. PubMed PMID: 25720452; PMCID: PMC4550575.
28. Kay R, Rosten P M, Humphries R K. CD24, a signal transducer modulating B cell activation responses, is a very short peptide with a glycosyl phosphatidylinositol membrane anchor. Journal of immunology (Baltimore, Md.: 1950). 1991; 147(4):1412-6. Epub 1991/08/15. PubMed PMID: 1831224.
29. Perry D, Sang A, Yin Y, Zheng Y Y, Morel L. Murine models of systemic lupus erythematosus. Journal of biomedicine & biotechnology. 2011; 2011:271694. Epub 2011/03/16. doi: 10.1155/2011/271694. PubMed PMID: 21403825; PMCID: PMC3042628.
30. Ge Y, Jiang C, Sung S S, Bagavant H, Dai C, Wang H, Kannapell C C, Cathro H P, Gaskin F, Fu S M. Cgnzl allele confers kidney resistance to damage preventing progression of immune complex-mediated acute lupus glomerulonephritis. J Exp Med. 2013; 210(11):2387-401. Epub 2013/10/09. doi: 10.1084/jem.20130731. PubMed PMID: 24101379; PMCID: PMC3804943.
1. Agnew L L, Kelly M, Howard J, et al. Altered lymphocyte heat shock protein 70 expression in patients with HIV disease. AIDS. 2003, 17(13):1985-8
2. Allers K, Fehr M, Conrad K, et al. Macrophages accumulate in the gut mucosa of untreated HIV-infected patients. J Infect Dis. 2014, 209(5):739-48
3. Anraku I, Rajasuriar R, Dobbin C, et al. Circulating heat shock protein 60 levels are elevated in HIV patients and are reduced by anti-retroviral therapy. PLoS One. 2012, 7(9):e45291
4. Appay V, Sauce D. Immune activation and inflammation in HIV-1 infection: causes and consequences. J Pathol. 2008, 214(2):231-41
5. Bai X F, Liu J Q, Liu X, et al. The heat-stable antigen determines pathogenicity of self-reactive T cells in experimental autoimmune encephalomyelitis. J Clin Invest. 2000, 105(9):1227-32
6. Bretz N P, Salnikov A V, Doberstein K, et al. Lack of CD24 expression in mice reduces the number of leukocytes in the colon. Immunol Lett. 2014, 161(1):140-8
7. Carl J W Jr, Liu J Q, Joshi P S, et al. Autoreactive T cells escape clonal deletion in the thymus by a CD24-dependent pathway. J Immunol. 2008, 181(1):320-8
8. Chase A, Zhou Y, Siliciano R F. HIV-1-induced depletion of CD4+ T cells in the gut: mechanism and therapeutic implications. Trends Pharmacol Sci. 2006, 27(1):4-7
9. Chen G Y, Chen X, King S, et al. Amelioration of sepsis by inhibiting sialidase-mediated disruption of the CD24-SiglecG interaction. Nat Biotechnol. 2011, 29(5):428-35
10. Chen G Y, Tang J, Zheng P, Liu Y. CD24 and Siglec-10 selectively repress tissue damage-induced immune responses. Science. 2009, 323:1722-5
11. Chen W, Han C, Xie B, et al. Induction of Siglec-G by RNA viruses inhibits the innate immune response by promoting RIG-I degradation. Cell. 2013, 152(3):467-78
12. Conrad K, Epple H J, Schürmann D, et al. Macrophages accumulate in the gut mucosa of untreated HIV-infected patients. J Infect Dis. 2014, 209(5):739-48
13. Deeks S G, Lewin S R, Havlir D V. The end of AIDS: HIV infection as a chronic disease. Lancet. 2013, 382(9903):1525-33
14. Deeks S G. Immune dysfunction, inflammation, and accelerated aging in patients on antiretroviral therapy. Top HIV Med. 2009, 17(4):118-23
15. Ding Y, Guo Z, Liu Y, et al. The lectin Siglec-G inhibits dentritic cell cross-presentation by impairing MHC class I-peptide complex formation. Nat Immunol. 2016, 17(10):1167-75.
16. Doitsh G, Galloway N L, Geng X, et al. Cell death by pyroptosis drives CD4 T-cell depletion in HIV-1 infection. Nature. 2014, 505(7484):509-14
17. Eckard A R, McComsey G A. The role of statins in the setting of HIV infection. Curr HIV/AIDS Rep. 2015, 12(3):305-12
18. Espigares E, Bueno A, Hernandez J, et al. Levels of HSP70 in HIV(+) patients in different viroimmunological states. J Med Virol. 2006, 78(3):318-23
19. Hahne M, Wenger R H, Vestweber D, Nielsen P J. The heat-stable antigen can alter very late antigen 4-mediated adhesion. J Exp Med. 1994 Apr. 1; 179(4):1391-5
20. Hsu D C, Sereti I. Serious Non-AIDS Events: Therapeutic Targets of Immune Activation and Chronic Inflammation in HIV Infection. Drugs. 2016, 76(5):533-49
21. Hunt P W, Martin J N, Sinclair E, et al. Valganciclovir reduces T cell activation in HIV-infected individuals with incomplete CD4+ T cell recovery on antiretroviral therapy. J Infect Dis. 2011, 203(10):1474-83
22. Kang J W, Kim S J, Cho H I, Lee S M. DAMPs activating innate immune responses in sepsis. Ageing Res Rev. 2015, 24(Pt A):54-65
23. Kelley C F, Kitchen C M, Hunt P W, et al. Incomplete peripheral CD4+ cell count restoration in HIV-infected patients receiving long-term antiretroviral treatment. Clint Infect Dis. 2009, 48:787-794
24. Kim T S, Gorski S A, Hahn S, et al. Distinct dendritic cell subsets dictate the fate decision between effector and memory CD8(+) T cell differentiation by a CD24-dependent mechanism. Immunity. 2014, 40(3):400-13
25. Kocsis J, Prohászka Z, Biró A, et al. Elevated levels of antibodies against 70 kDa heat shock proteins in the sera of patients with HIV infection. J Med Virol. 2003, 71(4):480-2;
26. Kristiansen G, Sammar M, Altevogt P. Tumour biological aspects of CD24, a mucin-like adhesion molecule. J Mol Histol. 2004, 35(3):255-62;
27. Kristoff J, Haret-Richter G, Ma D, et al. Early microbial translocation blockade reduces SIV-mediated inflammation and viral replication. J Clin Invest. 2014, 124:2802-2806
28. Kuwata T, Nishimura Y, Whined S, et al. Association of progressive CD4(+) T cell decline in SIV infection with the induction of autoreactive antibodies. PLoS Pathog. 2009, 5(4):e1000372
29. Lee K M, Ju J H, Jang K, et al. CD24 regulates cell proliferation and transforming growth factor β-induced epithelial to mesenchymal transition through modulation of integrin β1 stability. Cell Signal. 2012, 24(11):2132-42
30. Li D, Zheng L, Jin L, et al. CD24 polymorphisms affect risk and progression of chronic hepatitis B virus infection. Hepatology. 2009, 50(3):735-42
31. Li O, Chang X, Zhang H, et al. Massive and destructive T cell response to homeostatic cue in CD24-deficient lymphopenic hosts. J Exp Med. 2006, 203(7):1713-20
32. Liu Y, Jones B, Aruffo A, et al. Heat-stable antigen is a costimulatory molecule for CD4 T cell growth. J Exp Med. 1992, 175(2):437-45
33. Lotze M T, Tracey K J. High-mobility group box 1 protein (HMGB1): nuclear weapon in the immune arsenal. Nat Rev Immunol. 2005, 5(4):331-42
34. Malnati M S, Scarlatti G, Gatto F, et al. A universal real-time PCR assay for the quantification of group-M HIV-1 proviral load. Nat Protoc. 2008, 3(7):1240-8
35. Nowak P, Barqasho B, Sonnerborg A. Elevated plasma levels of high mobility group box protein 1 in patients with HIV-1 infection. AIDS. 2007, 21(7):869-71
36. Paiardini M, Müller-Trutwin M. HIV-associated chronic immune activation. Immunol Rev. 2013, 254:78-101
37. Pallikkuth S, Micci L, Ende Z S, et al. Maintenance of Intestinal Th17 Cells and Reduced Microbial Translocation in SIV-infected Rhesus Macaques Treated with Interleukin (IL)-21. PLoS Pathog. 2013, 9(7): 1003471
38. Rahman Z S. Impaired clearance of apoptotic cells in germinal centers: implications for loss of B cell tolerance and induction of autoimmunity. Immunol Res. 2011, 51(2-3):125-33
39. Rajasuriar R, Wright E, Lewin S R. Impact of antiretroviral therapy (ART) timing on chronic immune activation/inflammation and end-organ damage. Curr Opin HIV AIDS. 2015, 10(1):35-42
40. Rajasuriar R, Khoury G, Kamarulzaman A, et al. Persistent immune activation in chronic HIV infection: do any interventions work?. AIDS. 2013, 27(8):1199-208
41. Ravnskov U, Rosch P J, Sutter M C, Houston M C. Should we lower cholesterol as much as possible? BMJ. 2006, 3;332(7553):1330-2
42. Rawson P M, Molette C, Videtta M, et al. Cross-presentation of caspase-cleaved apoptotic self antigens in HIV infection. Nat Med. 2007, 13(12):1431-9
43. Sandler N G, Zhang X, Bosch R J, et al. Sevelamer does not decrease lipopolysaccharide or soluble CD14 levels but decreases soluble tissue factor, low-density lipoprotein (LDL) cholesterol, and oxidized LDL cholesterol levels in individuals with untreated HIV infection. J Infect Dis. 2014, 210:1549-54
44. Sattentau Q J, Stevenson M. Macrophages and HIV-1: An Unhealthy Constellation. Cell Host Microbe. 2016 Mar. 9; 19(3):304-10.
45. Shin J J, Lee E K, Park T J, Kim W. Damage-associated molecular patterns and their pathological relevance in diabetes mellitus. Ageing Res Rev. 2015, 24(Pt A):66-76.
46. Shive C L, Jiang W, Anthony D D, Lederman M M. Soluble CD14 is a nonspecific marker of monocyte activation. AIDS. 2015, 29(10):1263-5.
47. Steele A K, Lee E J, Manuzak J A, et al. Microbial exposure alters HIV-1-induced mucosal CD4+ T cell death pathways Ex vivo. Retrovirology. 2014, 11:14
48. Sun H, Pan Y, Wu R, et al. CD24 Ala57Val polymorphism is associated with spontaneous viral clearance in the HCV-infected Chinese population. Liver Int. 2015, 35(3):786-94
49. Tabb B, Morcock D R, Trubey C M, et al. Reduced inflammation and lymphoid tissue immunopathology in rhesus macaques receiving anti-tumor necrosis factor treatment during primary simian immunodeficiency virus infection. J Infect Dis. 2013, 207(6):880-92
50. Tian R R, Zhang M X, Zhang L T, et al. High immune activation and abnormal expression of cytokine contribute to death of SHIV89.6-infected Chinese rhesus macaques. Archives of virology. Arch Virol., 2015, 160(8):1953-66
51. Trøseid M, Sönnerborg A, Nowak P. High mobility group box protein-1 in HIV-1 infection. Curr HIV Res. 2011, 9(1):6-10.
52. Trøseid M, Nowak P, Nyström J, et al. Elevated plasma levels of lipopolysaccharide and high mobility group box-1 protein are associated with high viral load in HIV-1 infection: reduction by 2-year antiretroviral therapy. AIDS. 2010, 24(11):1733-7
53. Trøseid M, Lind A, Nowak P, et al. Circulating levels of HMGB1 are correlated strongly with MD2 in HIV-infection: possible implication for TLR4-signalling and chronic immune activation. Innate Immun. 2013, 19(3):290-7
54. Venereau E, De Leo F, Mezzapelle R, et al. HMGB1 as biomarker and drug target. Pharmacol Res. 2016, 111:534-44
55. Williams L A, McLellan A D, Summers K L, et al. Identification of a novel dendritic cell surface antigen defined by carbohydrate specific CD24 antibody cross-reactivity. Immunology. 1996, 89(1):120-5
56. Xia H J, Zhang G H, Ma J P, et al. Dendritic cell subsets dynamics and cytokine production in SIVmac239-infected Chinese rhesus macaques. 2010, Retrovirology. 7:102
57. Younas M, Psomas C, Reynes J, Corbeau P. Immune activation in the course of HIV-1 infection: Causes, phenotypes and persistence under therapy. HIV Med. 2016, (2):89-105

1. A method of treating HIV/AIDS, comprising administering a CD24 protein to a subject in need thereof.

2. The method of claim 1, wherein the CD24 protein comprises a mature human CD24 polypeptide or a variant thereof.

3. The method of claim 2, wherein the mature human CD24 polypeptide comprises the amino acid sequence set forth in SEQ ID NO: 1 or 2.

4. The method of claim 2, wherein the CD24 protein further comprises a protein tag, wherein the protein tag is fused at the N-terminus or C-terminus of the CD24 protein.

5. The method of claim 4, wherein the protein tag comprises a Fc region of a mammalian immunoglobulin (Ig) protein.

6. The method of claim 5, wherein the Ig protein is human.

7. The method of claim 6, wherein the Fc region comprises a hinge region and CH2 and CH3 domains of IgG1, IgG2, IgG3, IgG4, or IgA.

8. The method of claim 6, wherein the Fc region comprises a hinge region and CH2, CH3 and CH4 domains of IgM.

9. The method of claim 7, wherein the CD24 protein comprises the amino acid sequence set forth in SEQ ID NO: 6, ii, or 12.

10. The method of claim 9, wherein the amino acid sequence of the CD24 protein consists of the sequence set forth in SEQ ID NO: 6, 11, or 12.

11. The method of claim 1, wherein the CD24 protein is produced using a eukaryotic protein expression system.

12. The method of claim 11, wherein the expression system comprises a vector contained in a Chinese Hamster Ovary cell line or a replication-defective retroviral vector.

13. The method of claim 12, wherein the replication-defective retroviral vector is stably integrated into the genome of a eukaryotic cell.

14. The method of claim 1, wherein the CD24 protein is soluble.

15. The method of claim 1, wherein the CD24 protein is glycosylated.

16.-30. (canceled)
